(19) United States
(12) Patent Application Publication
(10) Pub. No.: US 2016/0000987 A1
(43) Pub. Date: Jan. 7, 2016

(54) DEVICE AND METHOD FOR PURIFYING VIRALLY INFECTED BLOOD

(71) Applicant: Aethlon Medical, Inc., San Diego, CA (US)

(72) Inventors: Richard H. Tullis, Encinitas, CA (US); Harold H. Handley, Encinitas, CA (US); James A. Joyce, San Diego, CA (US); Paul Duffin, San Diego, CA (US)

(21) Appl. No.: 14/856,361

(22) Filed: Sep. 16, 2015

Related U.S. Application Data
(63) Continuation of application No. 12/600,236, filed on May 12, 2011, now abandoned.
(60) Provisional application No. 60/938,432, filed on May 16, 2007.

(51) Int. Cl. A61M 1/36; B01D 69/02; B01D 15/22; B01D 69/08; B01D 61/02; B01D 15/38

(52) U.S. Cl. CPC A61M 1/362; B01D 61/027; B01D 15/3823; B01D 15/22; B01D 69/08; B01D 69/02; B01D 2325/02; A61M 2202/206

(57) ABSTRACT

The present invention relates to a method for using lectins that bind to pathogens having surface glycoproteins or fragments thereof which contain glycoproteins, to remove them from infected blood or plasma or other fluids in an extracorporeal setting. Accordingly, the present invention provides a methods and devices for reducing viral load or plaque forming units in blood or plasma from one or more infected individuals. A preferred embodiment of the method comprises passing the blood or plasma through a porous hollow fiber membrane wherein lectin molecules are disposed proximate to the membrane, collecting pass-through blood or plasma and optionally reinfusing the pass-through blood or plasma into the individual. Additionally, the present invention provides a methods and devices for the reduction of plaque forming units, cleared more rapidly and more efficiently than overall viral load.

### RELATED APPLICATIONS

This application is a continuation of U.S. application Ser. No. 12/600,236, filed May 12, 2011, which is the U.S. National Phase under 35 U.S.C. §371 of International Application No. PCT/US2008/063946, filed May 16, 2008 under the Patent Cooperation Treaty (PCT), which was published by the International Bureau in English, which designates the United States and claims the benefit of U.S. Provisional Application No. 60/938,432, filed May 16, 2007, the disclosures of which are hereby expressly incorporated by reference in their entireties.

### BACKGROUND OF THE INVENTION

1. Field of the Invention

The present invention relates to the field of therapeutic methodologies and devices for treating viral infections and removing viral particles from contaminated fluids.

2. Description of the Related Art

A large number of viruses have been described which are pathogenic for humans. Viruses such as ebola, marburg, smallpox, lassa, dengue, influenza (e.g. H5N1), measles, mumps, viral encephalitis (e.g. Japanese encephalitis), HIV, hepatitis, herpes, human cytomegalovirus (HCMV) and distemper are the etiological agents for debilitating and often incurable medical ailments. Aside from natural infection, the emerging threat of bioterror makes mass infections with these deadly agents ever more likely. Therapy is difficult for viral diseases as antibiotics have no effect on viruses and few antiviral drugs are known. In cases where drug treatments are available, the occurrence of resistant mutations and drug side effects often limit the effectiveness of therapy. Examples of such viruses include Hepatitis C and human immunodeficiency virus (HIV). The best way to prevent viral diseases is through vaccination; however, vaccines are unavailable for a large number of viruses, including many of the viruses listed above. Although there are vaccines present for others, many available vaccine strategies are either not fully effective, as in the case of Hepatitis B Virus, or present potentially life-threatening side-effects, such as the vaccine released and recalled for rotavirus. Further, where vaccines do exist they are predominantly preventive and largely ineffective once a viral infection becomes established in the host.

Dengue Virus is the etiological agent of both dengue fever and dengue hemorrhagic fever (DHF), acute febrile diseases found mainly in tropical environments. The World Health Organization estimates that there may be 50 million cases of dengue infection worldwide each year. Although both dengue fever and DHF cause similar symptoms, cases of DHF also show higher fever, haemorrhagic phenomena, thrombocytopenia and haemoconcentration. A small proportion of DHF cases lead to dengue shock syndrome (DSS) which has a high mortality rate.

No antiviral therapy exists for dengue fever or dengue hemorrhagic fever. The only available therapy is to encourage patients to keep up oral intake, especially of oral fluids. More severe cases require supplementation with intravenous fluids to prevent dehydration and significant hemoconcentration. Some cases of dengue fever and DHF require blood transfusions, as platelets are rapidly depleted. Acetaminophen is normally administered, but only to moderate the extreme pain and fever associated with the disease. There are no vaccines available to stem outbreak and the only therapy available is a sit and wait approach.

HIV infection is mediated by gp120, which binds to CD4 as well as to a surface chemokine receptor. Inside the cell the virion is uncoated and the viral RNA is reverse transcribed into double-stranded DNA. Proviral DNA enters the cell nucleus, integrates into the host genome and is transcribed into viral RNAs, which are translated into viral proteins. Mature virions are assembled and released from the cell by budding. (Fauci et al. Ann Intern Med 124(7): 654-663, 1996). A dying cell can also release all its contents including intact virions, and fragments thereof into the blood. Thus, circulating blood of HIV-infected individuals contains intact virions, and viral proteins, in particular toxic viral surface proteins.

The hallmark of AIDS is the loss of CD4+ T cells, which ultimately leaves the immune system unable to defend against opportunistic infections. While the mechanism through which HIV causes AIDS is imperfectly understood, the clinical data suggest that in addition to the loss of infected T-cells, a large number of uninfected T-cells are dying and that HIV derived envelope proteins appear to be intimately involved.

The major HIV envelope glycoprotein gp120 has been shown to have profound biological effects in vitro. Gp120 causes CD4+ T cells to undergo apoptosis and binding of gp120 to CD4+ cells in the presence of anti-envelope antibodies and complement opsoninizes the cells, targeting them for clearance. The combined effect is the destruction of uninfected immune cells. In addition, HIV envelope proteins have been implicated in HIV related hyper-gammaglobulinemia. In AIDS patients, gp120 levels have been measured at an average of 29 ng/ml which is orders of magnitude higher than the concentration of the virus.

Extracorporeal treatments provide a therapeutic modality which can be used to treat systemic disease. Extracorporeal perfusion of plasma over protein A, plasmapheresis and lymphapheresis have all been used as immunomodulatory treatments for HIV infection, and the thrombocytopenia resulting from it (Kiprov et al. Curr Stud Hematol Blood Transfus 57: 184-197, 1990; Mittelman et al. Semin Hematol 26(2 Suppl 1): 15-18, 1989; Snyder et al. Semin Hematol 26(2 Suppl 1): 31-41, 1989; Snyder et al. Aids 5(10): 1257-1260, 1991). These therapies are all proposed to work by removing immune complexes and other humoral mediators, which are generated during HIV infection. They do not directly remove HIV virus. Extracorporeal photopheresis has been tested in preliminary trials as a mechanism to limit viral replication (Bisaccia et al., J Acquir Immune Defic Syndr 6(4): 386-392, 1993; Bisaccia et al., Ann Intern Med 113(4): 270-275, 1990). However, none of these treatments effectively remove both virus and viral proteins.

Chromatographic techniques for the removal of HIV from blood products have been proposed. In 1997, Motomura et al., proposed salts of a sulfonated porous ion exchanger for removing HIV and related substances from body fluids (U.S. Pat. No. 5,667,684). Takashima and coworkers (U.S. Pat. No. 5,041,079) provide ion exchange agents comprising a solid substance with a weakly acidic or weakly alkaline surface for extracorporeal removal of HIV from the body fluids of a patient. Both are similar to the work of Porath and Janson (U.S. Pat. No. 3,925,152) who described a method of separating a mixture of charged colloidal particles, e.g. virus variants by passing the mixture over an adsorbent constituted of an insoluble, organic polymer containing amphoteric substituents composed of both basic nitrogen-containing groups and acidic carboxylate or sulphonate groups (U.S. Pat. No. 3,925,152). However, none of these chromatographic materials are selective for viruses and will clearly remove many other essential substances. Thus they are not useful for in vivo blood purification.

Immunosorptive techniques have also been proposed for the treatment of viral infections. In 1980, Terman et al. described a plasmapheresis apparatus for the extracorporeal treatment of disease including a device having an immunoadsorbent fixed on a large surface area spiral membrane to remove disease agents (U.S. Pat. No. 4,215,688). The device envisioned no method for directly treating blood and required the presence of an immunologically reactive toxic agent. In 1987 and 1988, Ambrus and Horvath described a blood purification system based on antibody or antigen capture matrices incorporated onto the outside surface of an asymmetric, toxin permeable membrane (U.S. Pat. Nos. 4,714,556; 4,787,974), however, no examples of pathogen removal were given therein. In 1991, Lopukhin et al. reported that rabbit antisera raised against HIV proteins, when coupled to Sepharose 4B or silica, could be used for extracorporeal removal of HIV proteins from the blood of rabbits which had been injected with recombinant HIV proteins (Lopukhin et al. Vestn Akad Med Nauk SSSR 11: 60-63, 1991). However, this strategy was inefficient as it required extracorporeal absorption of blood and did not provide for a mechanism to remove free HIV viral particles from the blood (Lopukhin et al., 1991, supra). U.S. Pat. No. 6,528,057 describes the removal of virus and viral nucleic acids using antibodies and antisense DNA.

Lectins are proteins that bind selectively to polysaccharides and glycoproteins and are widely distributed in plants and animals. Although many are insufficiently specific to be useful, it has recently been found that certain lectins are highly selective for enveloped viruses (De Clercq. et al Med Res Rev 20(5): 323-349, 2000). Among lectins which have this property are those derived from Galanthus nivalis in the form of Galanthus nivalis agglutinin (“GNA”), Narcissus pseudonarcissus in the form of Narcissus pseudonarcissus agglutinin (“NPA”) and a lectin derived from blue green algae Nostoc ellipsosporum called “cyanovirin” (Boyd et al. Antimicrob Agents Chemother 41(7): 1521-1530, 1997; Hammar et al. Ann N Y Acad Sci 724: 166-169, 1994; Kaku et al. Arch Biochem Biophys 279(2): 298-304, 1990). GNA is non-toxic and sufficiently safe that it has been incorporated into genetically engineered rice and potatoes (Bell et al. Transgenic Res 10(1): 35-42, 2001; Rao et al. Plant J 15(4): 469-477, 1998). These lectins bind to glycoproteins having a high mannose content such as found in HIV surface proteins (Chervenak et al. Biochemistry 34(16): 5685-5695, 1995). GNA has been employed in ELISA to assay HIV gp120 in human plasma (Hinkula et al. J Immunol Methods 175(1): 37-46, 1994; Mahmood et al. J Immunol Methods 151(1-2): 9-13, 1992; Sibille et al, Vet Microbiol 45(2-3): 259-267, 1995) and feline immunodeficiency virus (FIV) envelope protein in serum (Sibille et al. Vet Microbiol 45(2-3): 259-267, 1995). While GNA binds to envelope glycoproteins from HIV (types 1 and 2), simian immunodeficiency virus (SIV) (Gilljam et al. AIDS Res Hum Retroviruses 9(5): 431-438, 1993) and inhibits the growth of pathogens in culture, (Amin et al. Apmis 103(10): 714-720, 1995; Hammar et al. AIDS Res Hum Retroviruses 11(1): 87-95, 1995) such in vitro studies do not reflect the complex, proteinacious milieu found in HIV infected blood samples. It is therefore not known if lectins capable of binding high mannose glycoproteins in vitro would be able to bind such molecules in HIV infected blood samples. On the contrary, it is generally considered that the antibodies to gp120 typically present in individuals infected with HIV could sequester the high mannose glycoprotein sites to which lectins such as GNA bind.

Accordingly, although lectins are known to bind viral envelope glycoproteins, no previous technologies have demonstrated the ability to directly adsorb a wide spectrum of viruses, preferably enveloped viruses, from the blood using lectins in the setting of ex vivo dialysis or plasmapheresis. Therefore, there is an ongoing need for novel therapeutic approaches to the treatment of a broad spectrum of viral infections. In particular, there is a need for the development of novel approaches to reduce viral load, and live or infections virus in particular, so as to increase the effectiveness of other treatments and/or the immune response.

### SUMMARY OF THE INVENTION

The present invention utilizes lectins to bind, immobilize and retain whole virus, particularly infectious virus, as well as parts thereof, thus allowing a diminution of circulating virus and potential reduction of antigenic assault on the immune system. Of particular interest is the ability to preferentially remove live or infectious viral particles as compared to total viral load as measured, for example, by PCR.

One embodiment of the present invention is directed to a method and device using lectin to reduce the amount of viral plaque forming units, viral particles, and/or fragments thereof, in blood or plasma from one or more individuals infected with a lectin-binding virus, comprising the steps of: providing a lectin affinity device comprising a processing chamber having lectin disposed within the processing chamber, where the lectin binds viral plaque forming units, viral particles, and/or fragments thereof, in the blood or plasma and traps the viral plaque forming units, viral particles, and/or fragments thereof, in the processing chamber; transferring the blood or plasma into the chamber such that the viral plaque forming units, viral particles, and/or fragments thereof, contact the lectin and are bound thereto; removing the blood or plasma from the chamber, and optionally repeating the transferring and removing steps, where the blood or plasma is exposed to the lectin for no longer than 360 minutes.

Another embodiment of the present invention is directed to a method and device using lectin to reduce the amount of viral plaque forming units, viral particles, and/or fragments thereof, in blood or plasma from one or more individuals infected with a lectin-binding virus, comprising the steps of: providing a lectin affinity device comprising a processing chamber having lectin disposed within the processing chamber, where the lectin binds viral plaque forming units, viral particles, and/or fragments thereof, in the blood or plasma and traps the viral plaque forming units, viral particles, and/or fragments thereof, in the processing chamber; transferring the blood or plasma into the chamber such that the viral plaque forming units, viral particles, and/or fragments thereof, contact the lectin and are bound thereto; removing the blood or plasma from the chamber; and repeating the transferring and removing steps as often as required to remove at least 50% of the viral plaque forming units, viral particles, and/or fragments thereof, from the blood or plasma. In a preferred embodiment, the transferring and removing steps are repeated as often as required to remove about, at least, at least about, more than, more than about 55%, 60%, 65%, 70%, 75%, 80%, 85%, 90%, 91%, 92%, 93%, 94%, 95%, 96%, 97%, 98%, 99%, or 100% of the viral plaque forming units, viral particles, and/or fragments thereof, from the blood or plasma.

A further embodiment of the present invention is directed to a method and device using lectin to reduce the amount of viral plaque forming units, viral particles, and/or fragments thereof, in blood or plasma from one or more individuals infected with a lectin-binding virus, comprising: providing a lectin affinity device comprising a processing chamber having lectin disposed within the processing chamber, wherein the lectin binds viral plaque forming units, viral particles, and/or fragments thereof, in the blood or plasma and traps the viral plaque forming units, viral particles, and/or fragments thereof, in the processing chamber; transferring the blood or plasma into the chamber such that the viral plaque forming units, viral particles, and/or fragments thereof, contact the lectin and are bound thereto; removing the blood or plasma from the chamber; and repeating the transferring and removing steps as often as required until the remaining amount of viral plaque forming units, viral particles, and/or fragments thereof, is no greater than 1×10⁴/ml.

Another embodiment of the present invention is directed to a method and device using lectin to reduce the amount of viral plaque forming units, viral particles, and/or fragments thereof, in blood or plasma from one or more individuals infected with a lectin-binding virus, comprising the steps of: providing a lectin affinity device comprising a processing chamber having lectin disposed within the processing chamber, wherein the lectin binds viral plaque forming units, viral particles, and/or fragments thereof, in the blood or plasma and traps the viral plaque forming units, viral particles, and/or fragments thereof, in the processing chamber; transferring the blood or plasma into the chamber such that the viral plaque forming units, viral particles, and/or fragments thereof, contact the lectin and are bound thereto; removing the blood or plasma from the chamber; and repeating the transferring and removing steps as often as required until the amount of viral plaque forming units, viral particles, and/or fragments thereof, of the blood or plasma is reduced to a clinically relevant amount.

A method of using lectin to reduce the amount of viral load in blood or plasma from one or more individuals infected with a lectin-binding virus, comprising the steps of: providing a lectin affinity device comprising a processing chamber having lectin disposed within the processing chamber, where the lectin binds the virus, or lectin binding fragments thereof, in the blood or plasma and traps the virus, or lectin binding fragments thereof, in the processing chamber; transferring the blood or plasma into the chamber such that the virus, or lectin binding fragments thereof, contact the lectin and are bound thereto; removing the blood or plasma from the chamber, and optionally repeating the transferring and removing steps, where the blood or plasma is exposed to the lectin for no longer than 360 minutes.

A further embodiment of the present invention is directed to a method and device for treating an individual infected with a lectin-binding virus by reducing the amount of viral plaque forming units, viral particles, and/or fragments thereof, in the blood of the individual, the method comprising: identifying an individual infected with a lectin-binding virus; removing blood from the individual; providing a lectin affinity device comprising a processing chamber having lectin disposed within the processing chamber, where the lectin binds viral plaque forming units, viral particles, and/or fragments thereof, in the blood and traps the viral plaque forming units, viral particles, and/or fragments thereof, in the processing chamber; transferring the blood into the chamber such that the viral plaque forming units, viral particles, and/or fragments thereof, contact the lectin and are bound thereto; removing the blood from the chamber; returning the removed blood into the individual; repeating the removing, transferring, and returning steps until a volume of blood approximately equal to the total blood volume of the individual has been exposed to the lectin for no longer than 360 minutes.

In some of the embodiments, the chamber further comprises one or more porous hollow fiber membranes in the chamber, wherein lectin is disposed within an extrachannel or extralumenal space of the chamber proximate to an exterior surface of the membranes, and wherein the lectin binds the viral plaque forming units, viral particles, and/or fragments thereof, and traps them in the extrachannel space; wherein the method further comprises passing the blood or plasma through the hollow fiber membranes; and collecting pass-through blood or plasma. In some embodiments the method further comprises repeating the passing and collecting steps with the pass-through blood or plasma to further reduce the amount of the viral plaque forming units, viral particles, and/or fragments thereof, in the pass-through blood or plasma. In some embodiments the porous membranes allow passage of intact viral plaque forming units, viral particles, and/or fragments thereof, through the pores and exclude substantially all blood cells from passing through the pores.

In some of the embodiments, the blood or plasma can be exposed to the lectin for no longer than 60 minutes.

In some of the embodiments, the transferring and removing steps can be repeated.

In some of the embodiments, the removed blood or plasma can be reinfused into the individual.

In some of the embodiments, plasma contaminated with viral plaque forming units, viral particles, and/or fragments thereof, can be transferred into the chamber. In some of the embodiments, blood contaminated with viral plaque forming units, viral particles, and/or fragments thereof, can be transferred into the chamber.

In some of the embodiments, the processing chamber further comprises a porous membrane, the membrane configured such that the porous membrane allows passage of viral plaque forming units, viral particles, and/or fragments thereof, through the pores such that the viral plaque forming units, viral particles, and/or fragments thereof, contact the lectin, and the porous membrane excludes substantially all blood cells from passing through the pores, such that the blood cells do not contact the lectin. In some embodiments, the membrane has pores less than about 700 nm in diameter. In some embodiments, the membrane is a porous hollow fiber membrane. In some embodiments, the membranes have an inside diameter of about 0.3 mm and an outside diameter of about 0.5 mm.

In some of the embodiments, the lectin is attached to a substrate. In some embodiments, the substrate is selected from the group consisting of agarose, aminocelite, resins, silica, and proteins. In some embodiments, the substrate is a silica selected from the group consisting of glass beads, sand, and diatomaceous earth. In some embodiments, the substrate is a polysaccharide selected from the group consisting of dextran, cellulose and agarose. In some embodiments, the substrate is a protein comprising gelatin. In some embodiments, the substrate is a plastic selected from the group consisting of polystyrenes, polysuflones, polyesters, polyurethanes, polyacrylates and their activated and native amino and carboxyl derivatives. In some embodiments, the lectin is linked to the substrate by a linker. In some embodiments, the linker is a substrate, and/or is selected from the group consisting of gluteraldehyde, C2 to C18 dicarboxylates, diamines, dialdehydes, dihalides, and mixtures thereof.

In some of the embodiments, the lectin is selected from a group consisting of Galanthus nivalis agglutinin (GNA), Narcissus pseudonarcissus agglutinin (NPA), cyanovirin (CVN), ConconavalinA, Griffithsin and mixtures thereof. In some of the embodiments, the lectin is GNA.

In some of the embodiments, the lectin binds to a viral coat protein or a fragment thereof. In some of the embodiments, the virus is an enveloped virus. In some of the embodiments, the virus is a Category A enveloped virus. In some of the embodiments, the virus is a hemorrhagic fever virus. In some of the embodiments, the virus is selected from the group consisting of ebola, marburg, smallpox, lassa, dengue, rift valley, west nile, influenza A, influenza B, H5N1 influenza, measles, mumps, viral encephalitis, monkeypox, camelpox, vaccinia, HIV, HCV, hepatitis virus, human cytomegalovirus (HCMV) and distemper. In some of the embodiments, the virus is Dengue. In some of the embodiments, the virus is Influenza A or B. In some of the embodiments, the virus is H5N1 Influenza. In some of the embodiments, the virus is Ebola virus. In some of the embodiments, the virus is Monkeypox virus. In some of the embodiments, the virus is Vaccinia virus. In some of the embodiments, the virus is West Nile virus. In some embodiments, the virus is not HIV or HCV.

### BRIEF DESCRIPTION OF THE DRAWINGS

FIG. 1 is a schematic illustration of a longitudinal cross section of an embodiment of an affinity cartridge.

FIG. 2 is a schematic illustration of a horizontal cross section at plane 2 in FIG. 1.

FIG. 3 is an illustration of a channel from FIG. 2.

FIG. 4 is a graphical representation of the removal of viral protein from virus loaded physiological saline.

FIG. 5 is a graphical representation of the removal of viral fragments from virally infected human plasma.

FIGS. 6A and 6B illustrate the removal of native HIV on GNA Agarose FIG. 6A is a graphical representation of a plasmapheresis exponential curve where R²=0.90 (excluding one point at 22 hours). FIG. 6B is a graphical representation of a log plot of initial removal rate, where half time is about 0.9 hours.

FIG. 7 is a graphical representation of the removal of gp 120 from HIV+ blood.

FIG. 8 is a graphical representation of the removal of Hepatitis C virus infected blood.

FIG. 9 is a graphical representation of the average of three experiments measuring the removal of plaque forming units (pfu) and total viral load of Dengue Fever virus from cell culture supernatant.

FIG. 10 is a graphical representation measuring reduction of viral load of H5N1 Influenza virus from cell culture supernatant.

FIG. 11 is a graphical representation measuring reduction of viral load of recombinant 1918 Influenza virus from cell culture supernatant.

FIG. 12 is a graphical representation measuring reduction of viral load of Ebola Zaire virus from cell culture supernatant.

FIG. 13 is a graphical representation measuring reduction of viral load of Monkeypox virus from cell culture supernatant.

FIG. 14 is a graphical representation measuring reduction of viral load of Vaccinia virus from whole blood.

FIG. 15 is a graphical representation measuring reduction of viral load of West Nile virus from cell culture supernatant.

### DETAILED DESCRIPTION OF THE PREFERRED EMBODIMENT

The present invention relates to devices and methods for using lectins to remove pathogenic organisms and fragments thereof from infected blood or plasma, preferably in an extracorporeal setting. Accordingly, one embodiment of the present invention provides a method for reducing viral load or plaque forming units (pfu) in blood from an individual comprising the steps of obtaining blood or plasma from the individual, passing the blood or plasma through a porous hollow fiber membrane wherein lectin molecules which bind to glycoproteins, preferably high mannose glycoproteins, are immobilized within the porous exterior portion of the membrane, collecting pass-through blood or plasma, and optionally reinfusing the pass-through blood or plasma into the individual.

The term “viral load” as used herein refers to the amount of viral particles or toxic fragments thereof in a biological fluid, such as blood or plasma. “Viral load” encompasses all viral particles, infectious, replicative and non-infective, and fragments thereof. Therefore, viral load represents the total number of viral particles and/or fragments thereof circulating in the biological fluid. Viral load can therefore be a measure of any of a variety of indicators of the presence of a virus, such as viral copy number per unit of blood or plasma or units of viral proteins or fragments thereof per unit of blood or plasma.

The term “plaque forming units” or “pfu” as used herein refers to the amount of infectious virus particles in a biological fluid, such as blood or plasma. One plaque forming unit is equivalent to one infectious virus particle. A skilled artisan would recognize that viral plaque forming units are more critical to reduce than viral load. One important aspect of the present invention is its ability to reduce pfu/ml more efficiently than reducing viral load.

One skilled in the art would recognize that there are several ways to determine the number of plaque forming units in a particular sample. See, e.g., Lee H, and Jeong, Y S (2004) Comparison of Total Culturable Virus Assay and Multiplex Integrated Cell Culture-PCR for Reliability of Waterborne Virus Detection. Appl Environ Microbiol. 2004 June; 70(6): 3632-3636. In one particular assay, cells are grown on a flat surface until they form a monolayer of cells covering a bottle or dish. They are then infected with the target sample, or a particular dilution thereof. A plaque is produced when a virus particle infects a cell, replicates, and lyses, killing the cell. Surrounding cells are infected by the newly replicated virus and they too are killed. This process can repeat several times, such that sufficient numbers of neighboring cells are infected and lysed to form a cell-free hole within the monolayer of cells. The cells can be stained with a dye which stains only living cells. The dead cells in the plaque do not stain and appear as unstained areas on a colored background. Each plaque is the result of infection of one cell by one virus followed by replication and spreading of that virus. However, viruses that do not kill cells can not produce plaques and can contribute to the viral load without affecting the pfu count.

The term “high mannose glycoprotein” as used herein for the purpose of the specification and claims refers to glycoproteins having mannose-mannose linkages in the form of α-1->3 or α-1->6 mannose-mannose linkages. Some examples of lectins which bind glycoproteins including high mannose glycoproteins include, without limitation, Galanthus nivalis agglutinin (GNA), Narcissus pseudonarcissus agglutinin (NPA), cyanovirin (CVN), ConconavalinA, Griffithsin and mixtures thereof.

The term “exposed,” as used herein in the context of blood being “exposed” to any type of lectin-containing substrate, refers to any virus-containing portion of blood contacting a lectin-containing substrate. In some embodiments, the blood is exposed to the lectin-containing substrate for a specific amount of time. Exposure of the blood to the lectin-containing substrate, as used herein, refers to the total amount of time the blood is exposed to the lectin-containing substrate and not the amount of time blood is processed through the device.

The time of exposure is a function of the flow rate and the capacity of the lectin-containing substrate. For example, if the flow rate of a device is 10 ml/min and the capacity of the device is 10 ml, then running unprocessed blood for 30 minutes would expose 300 ml of blood to the lectin-containing substrate for 1 minute. For further illustration, if 30 ml of blood were recirculated over a device with the same flow rate and same capacity for 30 minutes, then the 30 ml of blood would be exposed to the lectin-containing substrate for 10 minutes. In some embodiments, the blood is exposed to a lectin-containing substrate is, is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 minutes. In other embodiments, the time the blood is exposed to a lectin-containing substrate is a range defined by any two times recited above.

In a preferred embodiment, the flow rate through the device is about 60 ml/min to about 400 ml/min. In a another preferred embodiment, the flow rate through the device is about 250 ml/min to about 400 ml/min. In some embodiments, the flow rate is, is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 ml/min., or a range defined by any two of these values. In some embodiments, the capacity of the device is 40 ml. Also contemplated are devices where the capacity is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 ml, or a range defined by any two of these values.

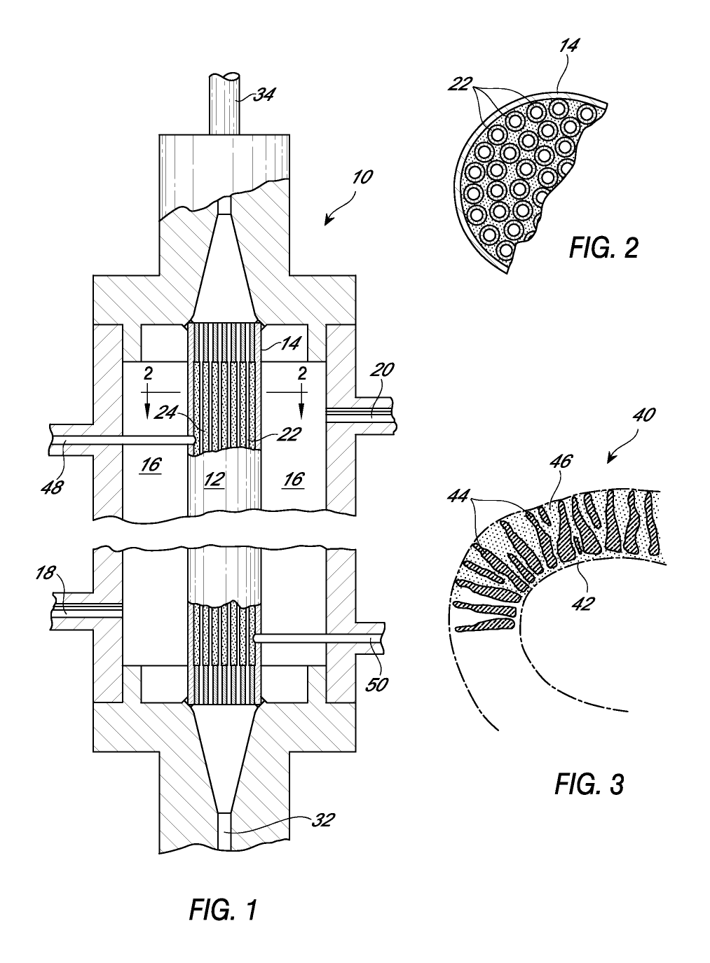

In a preferred embodiment, the method of the present invention is carried out by using an affinity cartridge such as the device illustrated in FIG. 1 and described below in greater detail. Devices of this general type are disclosed in U.S. Pat. Nos. 4,714,556, 4,787,974 and 6,528,057, the disclosures of which are incorporated herein by reference. In this device, blood is passed through the lumen of a hollow fiber membrane, wherein lectins are located in the extrachannel space of the cartridge, which form a means to accept and immobilize viruses and toxic and/or infectious fragments thereof. Thus, the device retains intact virions and viral glycoproteins bound by lectin while allowing other blood components to pass through the lumen.

Influenza A is primarily a respiratory virus with a low level of lethality and little indication of transmission via the blood. However, certain strains of the virus, such as H5N1 bird flu and the 1918 Spanish flu, have greatly increased mortality and morbidity. For these there is significant indication of blood borne viremia that can transmit the virus to other vital organs (de Jong, M, et al. N.E.J. Med 2006. 352:686; Zou, 2006 Transfus Med Rev 20(3):181-189). For these types of influenza infections, the affinity hemodialysis procedure would be efficacious. The invention can be used for the removal of any blood-borne viruses to which lectins bind. For example, viruses which can be cleared by the device include enveloped virus, Category A enveloped virus, ebola, marburg, smallpox, lassa, dengue, rift valley, west nile, influenza (e.g., H5N1), measles, mumps, viral encephalitis (e.g. Japanese encephalitis), monkeypox, camelpox, vaccinia, HIV, HCV, hepatitis virus, human cytomegalovirus (HCMV), distemper, swine pox, swine flu, siv, fiv, distemper, bird flu, sin nombre, yellow fever, herpes, SARS, sendai. In other embodiments, one or more viruses from the families of retroviridae, poxviridae paramyxoviridae (e.g., measles, mumps, sendai), orthomyxoviridae (e.g., bird flu, influenza), filoviridae (e.g., ebola, marburg), coronaviridae (e.g., SARS, encephalomyelitis), herpesviridae (e.g., herpes simplex, HCMV), rhabdoviridae (e.g., varicella stomatitis, rabies), and togavirus (e.g., rubella, semliki), are cleared. As used herein, “lectin-binding virus” is a virus which binds to or is bound by lectin. In some embodiments, the virus is not HIV or HCV.

In one embodiment, the device is used as a broad-spectrum treatment against bioterror threats. Smallpox is considered to be a Category “A” bioterror threat by the National Institute of Allergy and Infectious Diseases (NIAID). As research with human infectious smallpox is prohibited, MPV represents a primary model to study candidate therapies for smallpox virus. In one embodiment, concentrations of MPV are rapidly depleted from contaminated fluids, such as cell culture supernatant, plasma or blood, when circulated through the device.

Vaccinia is the “live pox-type virus” used in the smallpox vaccine. In one embodiment, high concentrations of vaccinia virus are rapidly depleted from contaminated fluids, such as cell culture supernatant, plasma or blood, when circulated through the device.

One embodiment of an affinity device, described in detail below with reference to FIGS. 1-3, includes multiple channels of hollow fiber membrane that forms a filtration chamber. An inlet port and an effluent port are in communication with the filtration chamber. The membrane is preferably an anisotropic membrane with the tight or retention side facing the bloodstream. The membrane is formed of any number of polymers known to the art, for example, polysulfone, polyethersulfone, polyamides, polyimides, and cellulose acetate. In other embodiments, the porous membrane is a sheet, rather than a channel. The sheet can be flat, or in some other configuration, such as accordion, concave, convex, conical, etc., depending on the device. In some embodiments, the membrane has pores with a mean diameter of, of about, of less than, of less than about, of more than, of more than about, 1950, 1900, 1850, 1800, 1750, 1700, 1650, 1600, 1550, 1500, 1450, 1400, 1350, 1300, 1250, 1200, 1150, 1100, 1050, 1000, 950, 900, 850, 800, 750, 700, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, or 85 nm, which will allow passage of intact viruses and viral particles and fragments (e.g., Rous Sarcoma Virus virions of 80 nm diameter, HCV of 50 nm), but not most blood cells. In other embodiments, the membrane has pores in a range between any two pore diameters recited above.

Preferably, the membrane has pores 200-500 nm in diameter, more preferably, the pore size is 600 nm, which will allow passage of intact viruses and viral particles and fragments (e.g., HIV virions of 110 nm diameter), but not most blood cells (red blood cells 10,000 nm diameter, lymphocytes 7,000-12,000 nm diameter, macrophages 10,000-18,000 nm diameter, thrombocytes 1000 nm). Optionally, by selecting a pore size that is smaller than the diameter of blood cells, the membrane excludes substantially all blood cells from passing through the pores and entering the extrachannel or extralumenal space of the device that contains the lectin. In some embodiments, a pore size is selected that is smaller than only some blood cell types.

A diagram of one embodiment of the device is shown in FIG. 1. The device comprises a cartridge 10 comprising a blood-processing chamber 12 formed of interior glass or plastic wall 14. Around chamber 12 is an optional exterior chamber 16. A temperature controlling fluid can be circulated into chamber 16 through port 18 and out of port 20. The device includes an inlet port 32 for the blood and an outlet port 34 for the effluent. The device also provides one or more ports 48 and 50, for accessing the extrachannel or extralumenal space in the cartridge. FIG. 2 is a schematic illustration of a horizontal cross section at plane 2 in FIG. 1. As shown in FIGS. 1 and 2, chamber 12 contains a plurality of membranes 22. These membranes preferably have a 0.3 mm inside diameter and 0.5 mm outside diameter. In some embodiments, the outside or inside diameter is 0.025 mm to 1 mm more preferably 0.1 to 0.5 mm more preferably 0.2 to 0.3 mm, as close to the outside diameter as allowed to minimize flow path length while still providing structural integrity to the fiber. FIG. 3 is a cross sectional representation of a channel 22 and shows the anisotropic nature of the membrane. As shown in FIG. 3, a hollow fiber membrane structure 40 is preferably composed of a single polymeric material which is formed into a tubular section comprising a relatively tight plasmapheresis membrane 42 and relatively porous exterior portion 44 in which can be immobilized lectins 46. During the operation of the device, a solution containing the lectins is loaded on to the device through port 48. The lectins are allowed to immobilize to the exterior 22 of the membrane in FIG. 2. Unbound lectins can be collected from port 50 by washing with saline or other solutions. Alternatively, the lectins can be bound to a substrate which is loaded into the extrachannel or extralumenal space, either as a dry substance (e.g. sand), or in solution or slurry.

In another embodiment, the device comprises a processing chamber having lectin disposed within the processing chamber, wherein said lectin binds viral particles or fragments in the blood or plasma, and traps them in the processing chamber. The blood or plasma can directly contact the lectins. In other embodiments, the device has a porous membrane which divides the chamber into one or more portions, such that the lectin is located in only a portion of the chamber. The preferred device utilizes hollow channel fiber membranes, but one or more sheets of membranes that divide the chamber are also contemplated. Where a membrane is used, the blood or plasma is filtered by the membrane, such that some portion of the blood or plasma is excluded from the portion of the chamber containing the lectin (e.g., blood cells or other large cells which cannot pass through the pores of the membrane).

In some embodiments, a device and method for reducing the viral load or pfu/ml in the blood or plasma by a therapeutically effective amount are provided. As used herein, the term “therapeutically effective amount” refers to a viral load or pfu/ml in the blood or plasma that halts or slows the progression of the infection, and slows and prevents the worsening of symptoms associated with the infection, and preferably improves and eliminates the infection or symptoms thereof. In some cases, reducing viral load or pfu/ml by or to a “therapeutically effective amount” can allow an infected individual's immune system to maintain or reduce the viral load or pfu/ml without further intervention. In some embodiments, “therapeutically effective amount” is an amount sufficient to render another treatment (e.g. a drugs, retroviral therapy, etc.) effective, or more effective. The “therapeutically effective amount” varies with different viruses and individuals, but can be readily determined by a skilled artisan. For example, for HIV infection current antiviral treatments have a target level of is no greater than about 1000 virus copies/ml, whereas Ebola infected monkeys are said to resolve disease on their own if the count can be reduced below 50,000 copies/ml (as measured by quantitative RT-PRC).

As evidenced by Table 1 below, the copies of virus per ml, varies from virus to virus. Just as the average viremia before clearance varies between viruses, so does the desired viral load or pfu/ml after clearance. In some embodiments, a “therapeutically effective amount,” or the desired viral load or pfu/ml after clearance is, is about, is less than, is less than about, is more than, is more than about 1×10⁹, 5×10⁸, 1×10⁸, 5×10⁷, 1×10⁷, 5×10⁶, 1×10⁶, 500,000, 450,000, 400,000, 350,000, 300,000, 250,000, 200,000, 150,000, 100,000, 90,000, 80,000, 70,000, 60,000, 50,000, 45,000, 40,000, 35,000, 30,000, 25,000, 20,000, 15,000, 10,000, 9000, 8000, 7000, 6000, 5000, 4000, 3000, 2000, 1000, 900, 800, 700, 600, 500, 450, 400, 350, 300, 250, 200, 190, 180, 170, 160, 150, 140, 130, 120, 100, 95, 90, 85, 80, 75, 70, 65, 60, 55, 50, 45, 40, 35, 30, 25, 20, 15, 10, 0. In some embodiments, the desired pfu/ml after clearance is a range defined by any two of the preceding numbers.

TABLE 1
Human Viral Infections

| | Viremia (copies per ml plasma)^{a} | | | | Reference |
|---|---|---|---|---|---|
| Viruses | Max | Mean | Survivable | Lethal | (number of patients) |
| Crimean Congo hemorrhagic fever | | 7.7 × 10⁵⁽¹⁾ | | | 1 (n = 1) |
| Dengue fever | 1.5 × 10⁷⁽⁵⁾ | 4.0 × 10⁷⁽¹¹⁾ | 4.0 × 10⁷⁽¹¹⁾ | | 5 (n = 20), 11 (n = 31) |
| | | 8 × 10⁵ | | | 1 (n = 1) |
| febrile | | 1.2 × 10^{5 (5)} | | | 5 (n = 20) |
| defevrescent | | Not detectable | | | 5 (n = 20) |
| Dengue hemorrhagic fever | 2 × 10⁹ | 3.2 × 10⁸⁽¹¹⁾ | 4.0 × 10⁷ | 3.2 × 10⁸⁽¹¹⁾ | 11 (n = 31) |
| febrile | | 1.5 × 10⁶⁽⁵⁾ | | | |
| defevrescent | | 4.3 × 10⁵⁽⁵⁾ | | | |
| Ebola | 1 × 10⁹⁽⁷⁾ | | 1 × 10⁷⁽⁷⁾ | 6.9 × 10⁸⁽¹⁾ | 1, 7 (n = 3) |
| Hepatitis C virus | | 3.2 × 10⁶ | | | National Genetics Inst |
| HIV | 2 × 10⁶⁽¹⁵⁾ | 2 × 10⁴⁽¹⁵⁾ | 1 × 10³⁽¹⁶⁾ | | 15 (n~100) |
| Influenza | not done | | | | |
| Lassa virus | 4 × 10⁹⁽¹⁾ | 7 × 10⁶⁽¹⁾ | | 4 × 10³⁽⁸⁾ | 1, 8 (n = 46) |
| | 1.0 × 10⁹⁽⁹⁾ | | | | 9 (n = 2) |
| Rift Valley fever | 1 × 10⁹⁽¹³⁾ | | | | 13 |
| Sin Nombre | | 1.3 × 10⁶⁽⁴⁾ | 6.3 × 10⁵⁽⁴⁾ | 5.0 × 10^{6 (4)} | 4 (n = 26) |
| Smallpox (Vaccinia) | 2 × 10⁵⁽¹²⁾ | | | | 12 (n = 10) |
| West Nile Virus | 1 × 10⁷⁽¹⁰⁾ | | | | 10 (n = 1) |
| Yellow fever | 1 × 10⁶⁽¹⁴⁾ | 4 × 10⁵⁽¹⁾ | | | 1 (n = 1) |

^{a}Viral load in copies per ml plasma is shown in scientific notation followed by the specific reference in parenthesis
References
1. Gunther, S (2002) J. Clinical Microbiology, 40 (7): 2323-2330.
2. Drosten, et al NEJM 348 (20): 1967-76, 2003
3. Zwiers, Miller, Baker, Kulesh, Jahrling and Huggins (USAMRIID)
4. Terajima, et at (1999) J Infect Dis 180: 2030.
5. Wang, WK et al (2003) Virology 20: 330.
6. Sanchez, et al (2004) J, Virol 18: 10370
7. Towner et al (2004) J. Virol 78: 4330
8. McCormick et al (1986) NEJM 314: 20
9. Schmitz et al (2002) Microbes Infect 4(1): 43-50
10. Paddock et al. (2006) CID 2006: 42 (June 1) 1527
11. Vaughn et at (2000) J Infect Dis 181: 2-9
12. Sharon et at (2003) JAMA Jun. 25, 2003 289 (24) 3295
13. Niklasson et at (1983) Journal of Clinical Microbiology 1026-1031 17(6)
14. Monath et al (2001) Lancet Infectious Diseases 1: 11-20

In one embodiment, the device is attached to an individual wherein the inlet port of the device is linked to the individual's vascular system, allowing blood to flow from the individual into the device, optionally with the assistance of a pump. In other embodiments, the blood from the individual is filtered or separated, allowing only the virus containing component to be exposed to a lectin-containing membrane. In some embodiments, the outlet port is also linked intravenously to the individual to allow the effluent blood to be reinfused into the individual. In one embodiment, the purified plasma is mixed with the cellular component before being reinfused into the individual. In another embodiment, the cellular component of the blood is reinfused into the individual separate from the effluent plasma.

In some embodiments, a volume equal to the total blood volume of the individual being treated is allowed to circulate at least once through the device. This does not necessarily mean that all of the blood in the individual passes through the device. As the blood is filtered and recirculated into the individual's blood stream, it is diluted by blood already present in the individual's blood stream. As such, it would be difficult to determine when all of the blood in the individual is circulated through the device. However, it can be determined when a volume equal to all of the individual's blood has been treated. Accordingly, the volume equal to the total blood volume of the individual being treated is defined as the total volume of blood run through the device being approximately equal to the estimated total blood volume present in the bloodstream of the individual being treated. For humans, the total blood volume for an average adult male weighing approximately 70 kg is between approximately 4 L and 5 L, (approximately 66 ml/kg) and the total volume of blood for an average adult female weighing approximately 50 kg is between approximately 3.0 L and 3.5 L (approximately 60 ml/kg). In some embodiments, a multiple of the total blood volume is treated. This multiple is, is about, is less than, is less than about, is more than, is more than about, 0.5, 1, 1.5, 2, 3, 4, 5, 6, 7, 8, 9, 10, 15, 20, 30, 40, 50, 60, 70, 80, 90, or 100, or a range defined by any two of these amounts.

The number of times the volume of blood being treated is required to be circulated through the device (treatment cycles) varies based on the replication rate of the virus being treated, the viral load or pfu/ml of the individual's blood, and the clearing rate of the device. The replication rate of viruses varies with each virus, but is known or can be determined by one skilled in the art. The viral load or pfu/ml within the individual's blood is dictated by the replication rate of the virus less the clearance rate of the virus. Further, the percentage of virus within the organs (non-blood borne), and the level of infectivity of the individual being treated influence the viral load, but can be ascertainable by a skilled artisan. The clearing rate of a particular device, although usually fixed across a broad spectrum of viruses, can vary. The clearing rate of a particular device is ascertainable by a person of ordinary skill in the art. Accordingly, the clinically relevant number of circulations is ascertainable without undue experimentation. The term “therapeutically effective number of circulations,” as used herein, refers to the number of circulations determined by a person of ordinary skill in the art to reduce the pfu/ml or viral load of the blood by or to a therapeutically effective amount.

In some embodiments, the number of times the blood or plasma being treated, which can be equal to the total blood volume of the individual being treated, or a multiple thereof, circulates through the device is, is about, is less than, is less than about, is more than, is more than about 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, 5, 4, 3, 2, 1. In some embodiments, the number of times the volume of blood equal to the total blood volume of the individual being treated circulates through the device is a range defined by any two numbers recited above.

Once the amount of blood or plasma to be processed and the number of circulations is determined, the time required for treatment is determined by the flow rate and capacity of the device. As such, the time required for a volume of blood or plasma to be processed on the device, or the amount of time an individual is treated by the device, can be determined by a skilled artisan. In some embodiments, the time required is, is about, is less than, is less than about, is more than, is more than about 600, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 minutes. In other embodiments, the time required for an individual to be processed on the device is a range defined by any two times recited above. In some embodiments, the individual's blood is continuously treated, and the device, or lectin portion of the device is periodically replaced.

In some embodiments, the process reduces the viral load or pfu/ml in the blood or plasma by, by about, by at least, by at least about, by more than, by more than about 99.9, 99.8, 99.5, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 45, 40, 35, 30, 25, 20, 15, or 10%. In other embodiments, the process reduces the viral load in the blood or plasma by a range defined by any two percentages recited above.

In some embodiments, the reduction in viral load or pfu/ml occurs within a limited amount of time. The amount of time required to reduce the viral load or pfu/ml to a desired level, or by a certain amount, is, is about, is less than, is less than about, is more than, is more than about 600, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 minutes.

As described in more detail in the Examples below, the devices and methods of the invention preferentially remove live viral particles (pfu) from blood or plasma more readily than other viral particles or fragments thereof. In some embodiments, the ratio of percent pfu clearance to percent viral load clearance is, is about, is less than, is less than about, is more than, is more than about, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.5:1, 7.0:1, 7.5:1, 8.0:1, 8.5:1, 9.0:1, 9.5:1, 10:1, 15:1, 20:1, 30:1, 40:1, 50:1, 75:1, 100:1, 125:1, 150:1, 175:1, or 200:1. In other embodiments, the ratio of pfu clearance to viral load clearance is a range defined by any two ratios recited above.

In one embodiment, blood having viral particles and/or fragments thereof is withdrawn from a patient and contacted with an membrane. In one preferred embodiment, the blood is separated into its plasma and cellular components. The plasma is then contacted with the lectins to remove the viral particles or fragments thereof by binding between viral high mannose glycoproteins and lectins. The plasma can then be recombined with the cellular components and returned to the patient. Alternatively, the cellular components can be returned to the patient separately. The treatment can be repeated periodically until a desired response has been achieved.

The technology to immobilize enzymes, chelators, and antibodies in dialysis-like cartridges has been developed (Ambrus et al., Science 201(4358): 837-839, 1978; Ambrus et al., Ann Intern Med 106(4): 531-537, 1987; Kalghatgi et al. Res Commun Chem Pathol Pharmacol 27(3): 551-561, 1980) and is incorporated herein by reference. These cartridges can be directly perfused with blood from patients through direct venous access, and returned to the patients without further manipulations. Alternatively, blood can be separated into plasma and cellular components by standard techniques. The cellular components can be combined with the plasma before reinfusing or the cellular components can be reinfused separately. Viral load can be assessed in the effluent from the cartridge by standard techniques such as ELISA and nucleic acid amplification and detection techniques. Prototypic cartridges have been used to metabolize excess phenylalanine (Kalghatgi et al., 1980, supra; Ambrus, 1978, supra) or to remove excess aluminum from patients' blood (Anthone et al. J Amer Soc Nephrol 6: 1271-1277, 1995). An illustration of preparing proteins for immobilization to the hollow fibers for the method of the present invention is presented in U.S. Pat. Nos. 4,714,556 and 4,787,974, 5,528,057.

For binding of lectins to the membrane, the polymers of the membrane are first activated, i.e., made susceptible for combining chemically with proteins, by using processes known in the art. Any number of different polymers can be used. To obtain a reactive polyacrylic acid polymer, for example, carbodiimides can be used (Valuev et al., 1998, Biomaterials, 19:41-3). Once the polymer has been activated, the lectins can be attached directly or via a linker to form in either case an affinity matrix. Suitable linkers include, but are not limited to, avidin, strepavidin, biotin, protein A, and protein G. The lectins can also be directly bound to the polymer of the membrane using coupling agents such as bifunctional reagents, or can be indirectly bound. In a preferred embodiment, GNA covalently coupled to agarose can be used to form an affinity matrix.

In some embodiments, the lectin is attached to a substrate instead of, or in addition to, the membrane. Suitable substrates include, but are not limited to, silica (e.g. glass beads, sand, diatomaceous earth) polysaccharides (e.g. dextran, cellulose, agarose), proteins (e.g. gelatin) and plastics (e.g. polystyrenes, polysuflones, polyethersulfones, polyesters, polyurethanes, polyacrylates and their activated and native amino and carboxyl derivatives). The lectin can be bound to the substrates through standard chemical means, either directly, or through linkers such as C2 to C>20 linear and branched carbon chains, as well as the plastics, proteins and polysaccharides listed above. For most synthetic purposes, C18 is the preferred upper limit but the chains can be added together for solubility reasons. Preferred linkers include: C2 to C18 dicarboxylates, diamines, dialdehydes, dihalides, and mixtures thereof (e.g. aminocarboxylates) in both native and activated form (e.g. disuccinimidyl suberimidate (DSS)). In some embodiments, one or more substrates can be used as linkers, alone or in combination with the substances listed as linkers. For example, dextran can be attached to sand, and additional linkers can then optionally be added to the dextran.

In certain embodiments, the virus cleared in any of the above recited embodiments does not include at one or more of the viruses selected from the group consisting of ebola, marburg, smallpox, lassa, dengue, rift valley, west nile, influenza (e.g., H5N1), measles, mumps, viral encephalitis (e.g. Japanese encephalitis), monkeypox, camelpox, vaccinia, HIV, HCV, hepatitis virus, human cytomegalovirus (HCMV), distemper, swine pox, swine flu, siv, fiv, distemper, bird flu, sin nombre, yellow fever, herpes, SARS, sendai.

As used herein, individual or subject, refers to any animal whose blood or other bodily fluid is being treated, and is not limited to humans. Individuals or subjects include all animals, including but not limited to primates such as monkeys and apes, dogs, cats, rats, mice, rabbits, pigs, and horses.

Although the embodiments described herein refer to removal of virus particles or fragments thereof from blood or plasma, one of skill in the art will appreciate that the device and methods described herein can be used with other fluids, such as other bodily fluids, cell culture supernatants, buffers, etc., which are contaminated with or contain lectin-binding virus or viral particles.

U.S. patent application Ser. No. 10/760,810, issued as U.S. Pat. No. 7,226,429, and the articles, patents, and other printed materials referred to herein, are hereby incorporated by reference in their entirety, and particularly for the material referred to above.

The following examples are presented to illustrate embodiments of this invention and are not intended to be restrictive.

### Example 1

This Example demonstrates the preparation of an affinity matrix using GNA covalently coupled to agarose using cyanogen bromide. Cyanogen bromide (CNBr) activated agarose was used for direct coupling essentially according to Cuatrecasas, et al (Cuatracasas et al. Proc Natl Acad Sci USA 61(2): 636-643, 1968). In brief, 1 ml of GNA at a concentration of 10 mg/ml in 0.1M NaHCO₃ pH 9.5 was added to 1 ml CNBr activated agarose (Sigma, St. Louis, Mo.) and allowed to react overnight in the cold. When the reaction was complete, unreacted materials were aspirated and the lectin coupled agarose washed extensively with sterile cold PBS. The lectin agarose affinity matrix was then stored cold until ready for use. Alternatively, GNA agarose is available commercially from Vector Labs (Burlingame, Calif.)

### Example 2

This Example demonstrates preparation of the lectin affinity matrix using GNA covalently coupled to glass beads via Schiff's base and reduction with cyanoborohydride. The silica lectin affinity matrix was prepared by a modification of the method of Hermanson (Hermanson. Bioconjugate Techniques: 785, 1996). GNA lectin was dissolved to a final protein concentration of 10 mg/ml in 0.1M sodium borate pH 9.5 and added to aldehyde derivatized silica glass beads (BioConnexant, Austin Tex.). The reaction is most efficient at alkaline pH but will go at pH 7-9 and is normally done at a 2-4 fold excess of GNA over coupling sites. To this mixture was added 10 μl 5M NaCNBH₃ in 1N NaOH (Aldrich, St Louis, Mo.) per ml of coupling reaction and the mixture allowed to react for 2 hours at room temperature. At the end of the reaction, remaining unreacted aldehyde on the glass surfaces are capped with 20 μl 3M ethanolamine pH 9.5 per ml of reaction. After 15 minutes at room temperature, the reaction solution was decanted and the unbound proteins and reagents removed by washing extensively in PBS. The matrix was the stored in the refrigerator until ready for use.

### Example 3

This Example demonstrates preparation of GNA covalently coupled to aminocelite using glutaraldehyde. Aminocelite was prepared by reaction of celite (silicate containing diatomaceous earth) by overnight reaction in a 5% aqueous solution of aminopropyl triethoxysilane. The aminated celite was washed free of excess reagent with water and ethanol and dried overnight to yield an off white powder. One gram of the powder was then suspended in 5 ml 5% glutaraldehyde (Sigma) for 30 minutes. Excess glutaraldehyde was then removed by filtration and washing with water until no detectable aldehyde remained in the wash using Schiffs reagent. The filter cake was then resuspended in 5 ml of Sigma borohydride coupling buffer containing 2-3 mg/ml GNA and the reaction allowed to proceed overnight at room temperature. At the end of the reaction, unreacted GNA was washed off and the unreacted aldehyde aminated with ethanolamine as described. After final washing in sterile PBS, the material was stored cold until ready for use.

### Example 4

This Example demonstrates the preparation of an exemplary lectin plasmapheresis device. Small volume filter cartridges (Glen Research, Silverton, Va.) were prepared containing 0.2 ml lectin resin, sealed and equilibrated with 5-10 column volumes sterile PBS. The cartridges were used immediately.

### Example 5

This Example demonstrates preparation of a GNA lectin affinity hemodialysis device. The viral hemodialysis device was made by pumping a slurry of particulate immobilized GNA on agarose beads or celite in sterile PBS buffer into the outside compartment of a hollow-fiber dialysis column using a syringe. For blood samples up to 15 mls, Microkros polyethersulfone hollow-fiber dialysis cartridge equipped with Luer fittings (200μ ID×240μ OD, pore diameter 200-500 nm, ˜0.5 ml internal volume) obtained from Spectrum Labs (Rancho Dominguez, Calif.) were used. Cartridges containing the affinity resin were equilibrated with 5-10 column volumes sterile PBS.

### Example 6

This Example demonstrates removal of HIV gp120 from physiological saline using an affinity plasmapheresis device. The plasmapheresis device described in Example 4 was equilibrated with 5-10 column volumes sterile PBS. A sample ˜1.5 ml containing gp120 (typically 500 ng/ml) was circulated over the column at a flow rate of 0.5-0.6 ml/min at room temperature. The circulating solution was tested at various time intervals for the presence of gp120 and gp120 immune complexes where appropriate.

Quantitative ELISA assays for HIV-1 gp120 were performed using a modification of the method of Weiler (Weiler et al. J Virol Methods 32(2-3): 287-301, 1991). GNA/NPA plates were prepared on Greiner C bottom plates by adding 100 μl protein (1-100 μg/ml each of GNA and NPA in PBS) to each well and incubating 2 hours at 37° C. The plates were then washed in PBST (PBS containing 0.01% Tween 20) and blocked in Casein blocking buffer for 1 hour at 37° C. Plates not used immediately were stored for up to 2 weeks at 4° C.

For detection of free gp120, 100 μl samples of test solutions were incubated for 1-2 hours at 37° C. After capture, plates were washed in PBS and 100 μl of the appropriate horse radish peroxidase (HRP) labeled anti-gp120 antibody (1:2500 in blocking buffer) was added. After incubation for 1 hour at 37° C. the antiserum was aspirated and the plates washed 4 times with 300 μl PBSTA and the bound HRP detected with stabilized tetramethylbenzidine (TMB) substrate (BioFx). For the determination of immune complex and immune complex formation, after capture, plates were washed in PBS and 100 μl of affinity purified HRP labeled sheep anti-human IgG antibody (1:2500 in blocking buffer) was added. After incubation for 1 hour at 37° C. the antiserum was aspirated and the plates washed 4×300 μl PBSTA. Bound HRP was detected with tetramethylbenzidine (TMB) (BioFx).

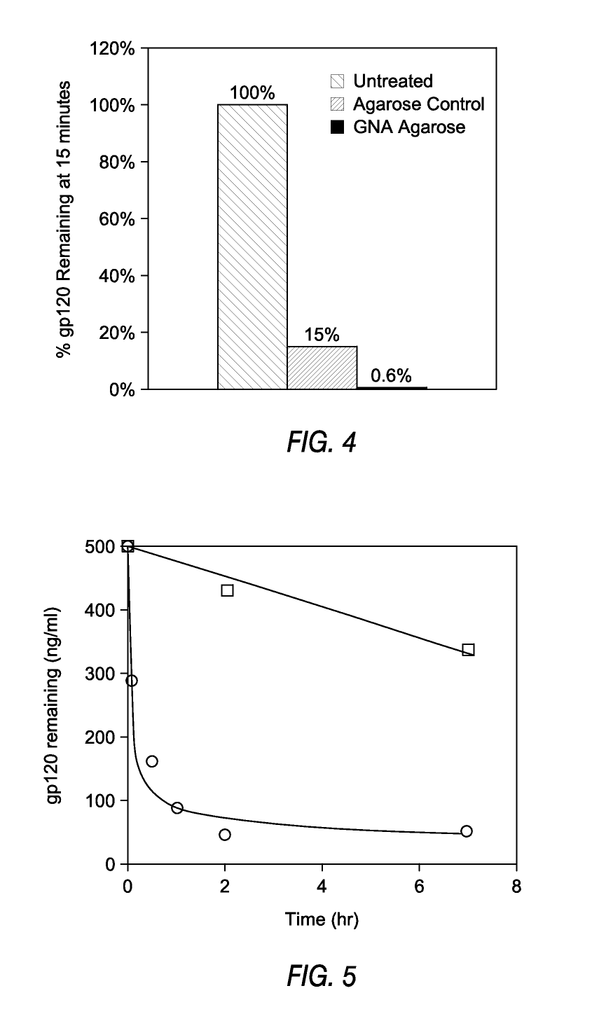

FIG. 4 shows that GNA agarose removed gp120 from buffer solution with 99% efficiency in <15 minutes. Because gp120 is a heavily glycosylated protein which can bind non-specifically to a variety of surfaces, it is not surprising that the control column also bound 85% of the input gp120. These results were previously presented in U.S. patent application Ser. No. 10/760,810, issued as U.S. Pat. No. 7,226,429.

### Example 7

This Example demonstrates the removal of HIV gp120 from infected plasma using a lectin affinity plasmapheresis device. The plasmapheresis device described in Example 4 was equilibrated with 5-10 column volumes sterile PBS. A plasma sample of about 1.5 ml containing gp120 (typically 500 ng/ml) was circulated over the column at a flow rate of 0.5-0.6 ml/min at room temperature. The circulating solution was tested at various time intervals for the presence of gp120 and gp120 immune complexes where appropriate as in Example 6.

Since anti-gp120 antibodies are typically abundant in HIV+ plasma, removal of gp120 from infected plasma might be expected to be more difficult than removal from simple buffer solutions. In part due to these antibodies, gp120 detection in HIV+ plasma and blood typically shows at best low amounts of gp120. In order to measure removal it was therefore necessary to add gp120 to infected patient plasma to provide a sample for measurement. ELISA measurement of the sample confirmed that all of the added gp120 in this sample was complexed with anti-gp120 antibodies (data not shown).

FIG. 5 shows that the GNA agarose affinity resin effectively removed gp120 in immune complexes from HIV infected plasma samples. Removal was rapid with an apparent half reaction time of 20 minutes. A portion of the gp120 signal was not removed (˜10% of the initial gp120 immune complex) even after 7 hours and appeared to represent background binding of IgG in the assay. These results were previously presented in U.S. patent application Ser. No. 10/760,810, issued as U.S. Pat. No. 7,226,429.

### Example 8

This Example demonstrates removal of HIV virions from infected plasma using GNA plasmapheresis. An HIV infected plasma sample (ER8-03030-0002 native HIV, Boston Biomedica, Boston Mass.) containing 100,000 copies per ml (cpm) of the virus was circulated over a 0.2 ml GNA agarose column described in Example 4. At intervals, 250 μl aliquots of the plasma were taken and the viral RNA extracted using TRI-LS reagent according to the manufacturers instructions (MRC Corporation). HIV viral RNA was then quantitated using real time RT PCR and an Access 1 step reagent set from Promega (Madison, Wis.) in 25 μl reaction volumes containing 400 nM SK432 and SK461 gag gene primers, Sybr green (1:10,000), 1×SCA blocking buffer, 3 mM MgCl₂, 400 uM dNTPs and 10 μl of unknown RNA or HIV-1 RNA from armored RNA standards (Ambion Austin Tex.). Amplification and reaction times were: RT (45 minutes at 48° C.) and PCR 40 cycles (94° C./15 sec; 62° C./30 sec; 72° C./60 sec; 83° C./read) in a SmartCycler real time thermocycler (Cepheid, Sunnyvale, Calif.) essentially according to the manufacturers instructions. When necessary for confirmation of amplification, 10 μl aliquots of the amplification mix were subjected to agarose gel electrophoresis 2%(w/v) (Sigma, molecular biology grade) in 0.5× TBE buffer pH 8.3 containing 0.25 μg/ml ethidium bromide for 45 minutes at 120 VDC at room temperature. Gels were photographed on a UV transilluminator with the images subsequently digitized and analyzed using ImageJ.

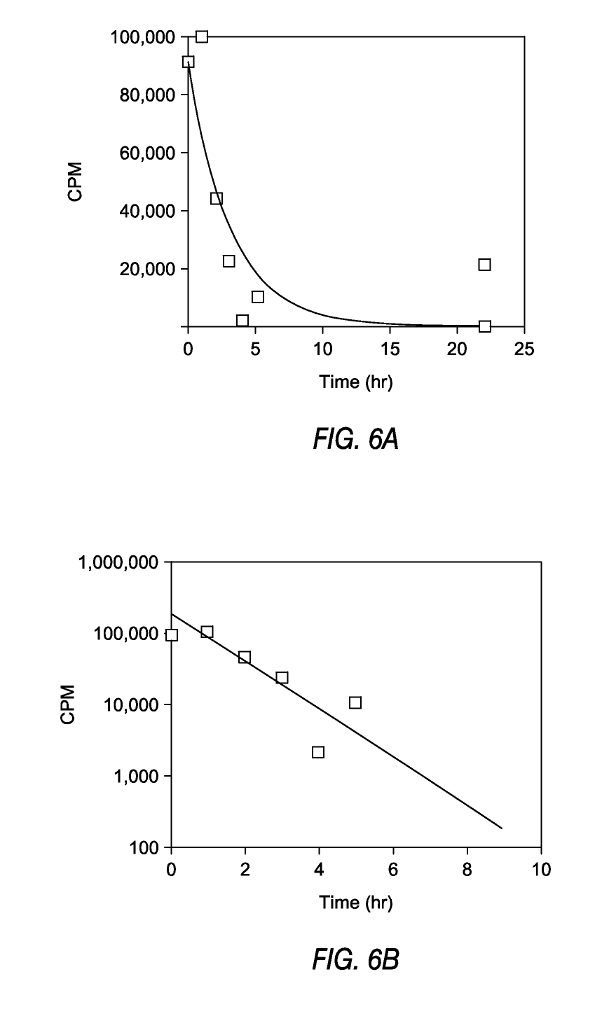

FIGS. 6A and 6B show that GNA agarose effectively removes HIV virions from infected plasma. FIG. 6A is a linear plot of the data curve fit to a exponential decay (R²=0.9). The curve predicts essentially quantitative removal of HIV in about 10 hours. FIG. 6B is a log plot of the HIV removal rate which gives an estimate of 0.9 hours as the half time of HIV removal. Virus removal appears first order as expected for GNA in excess over virus. CPM indicates HIV copies/ml. These results were previously presented in U.S. patent application Ser. No. 10/760,810, issued as U.S. Pat. No. 7,226,429.

### Example 9

This Example demonstrates removal of gp120 from HIV infected blood using a GNA lectin affinity hemodialysis device. Since most HIV+ plasma samples have low or undetectable amounts of gp120, simulated HIV infected blood samples were prepared by mixing 5 ml type O+ fresh packed red cells with 5 ml HIV infected plasma (typically 105 cpm) to which was added sufficient gp120 IIIB to make the sample 100 ng/ml

The affinity hemodialysis devices described in Example 5 were equilibrated with 5-10 column volumes sterile PBS. A control column containing only Sepharose 4B was prepared as a control. The infected blood sample ˜10 ml containing gp120 was recirculated over the column at a flow rate of 0.9 ml/min at 37° C. using a Masterflex roller pump (1 rpm) and Pharmed 6485-16 silicon tubing. The circulating solution was tested at various time intervals for the presence of free gp120 after acid denaturation and neutralization to disrupt immune complexes.

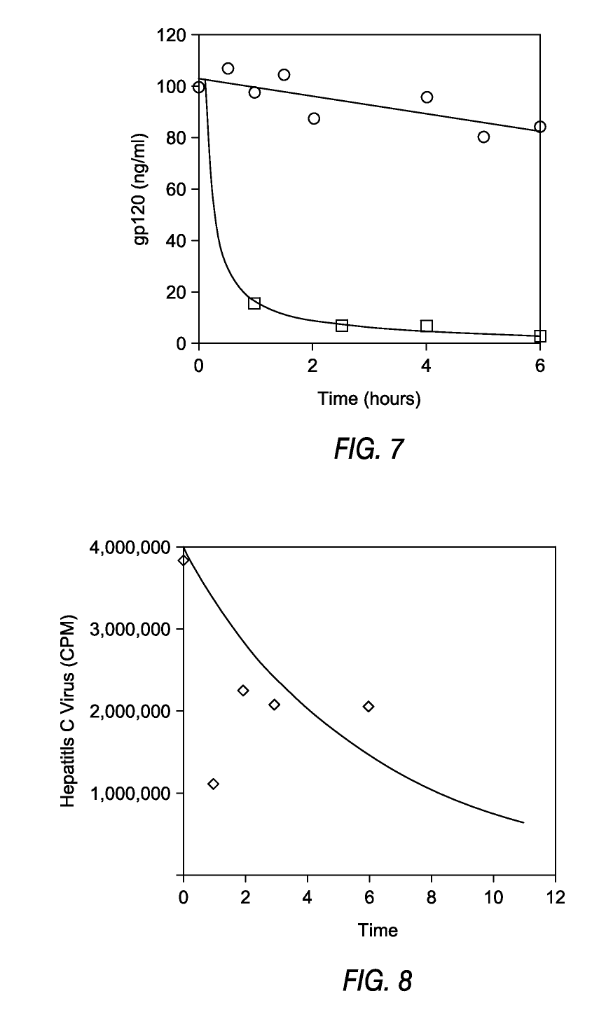

FIG. 7 shows that as the blood samples were recirculated over the cartridge, the initial gp120 of 100 ng/ml was reduced to background levels in 4 to 6 hours (apparent t_{1/2}=22 min). The control cartridge removed gp120 very slowly. These results were previously presented in U.S. patent application Ser. No. 10/760,810, issued as U.S. Pat. No. 7,226,429.

### Example 10

This example demonstrate removal of HCV from infected blood using GNA lectin affinity hemodialysis. The lectin affinity hemodialysis devices described in Example 4 were equilibrated with 5-10 column volumes sterile PBS. HCV infected blood samples were prepared by mixing 1 ml type O+ fresh packed red cells with 1 ml HIV infected plasma (typically 10⁵ cpm). The infected blood sample was recirculated over the column at a flow rate of 0.5 ml/min at room temperature using a Masterflex roller pump (1 rpm) and Pharmed 6485-16 tubing. The circulating solution was tested at various time intervals for the presence of HCV viral RNA.

Viral RNA was isolated using TRI-LS (MRC Corporation) from 100 μl of plasma according to the manufacturers instructions. HCV viral RNA was then measured by quantitative RT PCR performed using an Improm II reagent set from Promega (Madison, Wis.) in 25 ul reaction volumes containing 400 nM EY80 and EY78 HCV specific primers, Sybr green (1:10,000), 1×SCA blocking buffer, 3 mM MgCl₂, 400 uM dNTPs, 0.2 units/ul each of Tfl polymerase and AMV reverse transcriptase. Typically 50 ul of the mix was used to dissolve RNA isolated from 100 μl plasma and the mix split into two identical duplicate samples. Amplification and reaction times were: RT (45 minutes at 48° C.) and PCR 40 cycles (94° C./15 sec; 62° C./30 sec; 72° C./60 sec; 87° C. readout) in a SmartCycler real time thermocycler (Cepheid, Calif.) essentially according to the manufacturers instructions. The amount of viral RNA was estimated by comparison to the signal strength of the viral RNA standards in the initial phase of the amplification reaction (Cₜ=20).

FIG. 8 shows that as the blood was recirculated over the cartridge, the initial HCV was reduced about 50% in 3 hours (apparent t_{1/2}=3 hours). The curve fit reasonably well to an exponential decay. These results were previously presented in U.S. patent application Ser. No. 10/760,810, issued as U.S. Pat. No. 7,226,429.

### Example 11

Cell culture supernatants (5 ml) from Dengue Virus infected Vero cells were circulated through a GNA affinity matrix cartridge at 1.5 ml/min. Samples were collected prior to the start of circulation and at various time points after circulation. The Experiment was conducted 4 times with Experiment 2 failing due to operator error. The amount of viral load (viral RNA) and the amount of pfu (infectious virus) in each collected sample was determined by real-time quantitative reverse transcriptase polymerase chain reaction (qRT-PCR) and conventional plaque assay, respectively. The collected data is presented in Table 2.

TABLE 2

| | | | RNA | |
|---|---|---|---|---|
| | pfu/ml | % pfu/ml | copies/ml | % copies/ml |
| Experiment 1 | (plaque assay) | Reduction | (qRT-PCR) | Reduction |
| Before | 1.8 × 10⁶ | | 2.8 × 10¹⁰ | |
| Circulation | | | | |
| 30 min. after | 5.7 × 10³ | 99.6% | 1.9 × 10¹⁰ | 32.2% |
| Circulation | | | | |
| 60 min. after | 4.7 × 10³ | 99.7% | 1.5 × 10¹⁰ | 46.4% |
| Circulation | | | | |

Experiment 2: Failed due to operator error.

| | pfu/ml | | RNA | |
|---|---|---|---|---|
| | (plaque | % pfu/ml | copies/ml | % copies/ml |
| | assay) | Reduction | (qRT-PCR) | Reduction |
| Experiment 3 | | | | |
| Before Circulation | 6.4 × 10⁵ | | 1.5 × 10¹⁰ | |
| 60 min. after | 4.6 × 10⁴ | 93% | 1.3 × 10¹⁰ | 13% |
| Circulation | | | | |
| Experiment 4 | | | | |
| Before Circulation | 7.1 × 10⁵ | | 1.7 × 10¹⁰ | |
| 1 hr. after | 7.7 × 10⁴ | 89% | 1.2 × 10¹⁰ | 28% |
| Circulation | | | | |
| 2 hrs. after | 6.9 × 10⁴ | 90% | 7.9 × 10⁹ | 53% |
| Circulation | | | | |
| 3 hrs. after | 4.0 × 10⁴ | 94% | 4.5 × 10⁹ | 73% |
| Circulation | | | | |
| 4 hrs. after | 2.5 × 10⁴ | 96% | 2.0 × 10⁹ | 88% |
| Circulation | | | | |
| 5 hrs. after | 1.3 × 10⁴ | 98% | 1.5 × 10⁹ | 91% |
| Circulation | | | | |
| 6 hrs. after | 5.0 × 10³ | 99% | 1.1 × 10⁹ | 93% |
| Circulation | | | | |

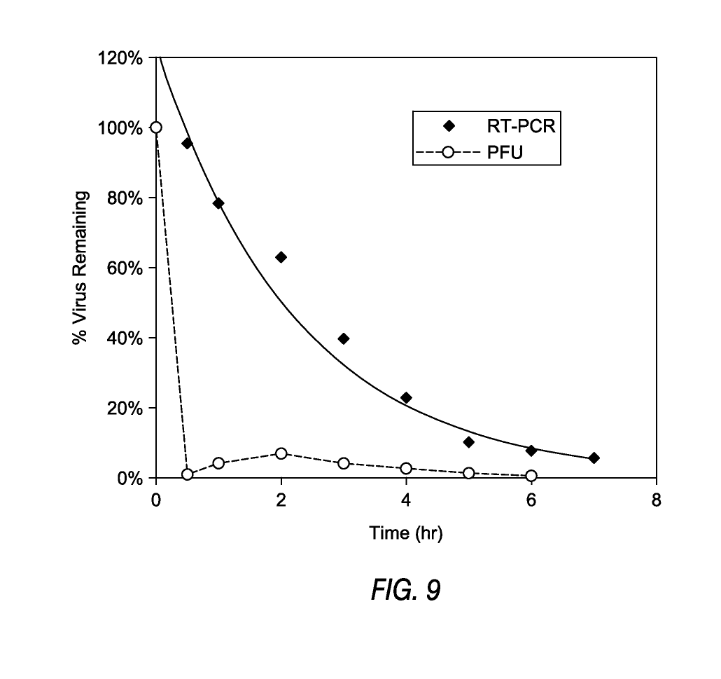

Importantly, live infectious Dengue Virus is removed more efficiently than total virus RNA. In Experiment 1, while only 32% of total viral load as measured by PCR was removed in, ½ hour, 99.6% of pfu, as indicated by plaque assay, was removed in the same time frame. This observation was confirmed at 1 hour. In Experiments 3 and 4, it is clear that live infectious Dengue Virus is removed more efficiently than virus RNA. FIG. 9 is a graphical depiction of the average of Experiments 1, 3, and 4 and demonstrates the greater efficiency with which plaque forming units are cleared from the blood after circulation through the device relative to the removal of viral load as measured by RT-PCR. The clearance of pfu live virus from biological fluids is thus more pronounced than would be indicated by assays designed to measure viral load without regard to the prevalence of infectious particles.

### Example 12

Cell culture supernatants (5 ml) from H5N1 infected cells (10⁶ to 10⁷ copies/mil) were circulated through a GNA affinity matrix cartridge at 1 ml/min (HP Treated). Samples were taken prior to the start and after 0, 1, 2, 4, 6 hour of recirculation. The recirculation were continued overnight (18-24 hr) for the final sample. Untreated samples to control for virus decomposition were taken at 6 hrs and overnight. The amount of viral RNA in the sample was determined before and after circulation through the cartridge by quantitative real time RT PCR using a modified version of the protocol outlined in Example 8 for use on cell culture supernatant. All PCR samples were determined in triplicate.

The collected data is presented in Table 3. The half-time was 57 minutes (initial 3.25×10⁶ cpm).

TABLE 3
H5N1 (initial 3.25 × 10⁶ cpm)

| | HP Treated | Control |
|---|---|---|
| Time (h) | % Initial | % Initial |
| 0 | 100% | 100% |
| 1 | 41% | — |
| 2 | 18% | — |
| 4 | 18% | — |
| 6 | 11% | — |
| 20 | 3% | 21% |

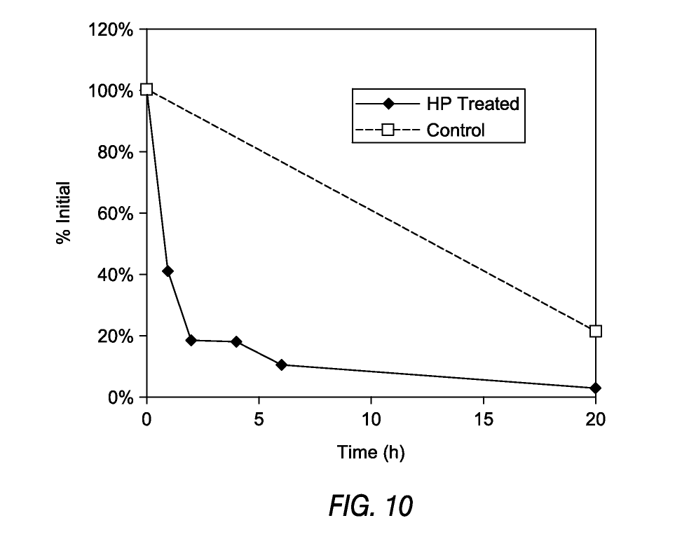

FIG. 10 is a graphical depiction of the average of the three experiments.

### Example 13

Cell culture supernatants (5 ml) from 1918 Influenza virus infected cells (10⁹ to 10¹⁰ copies/ml) were circulated through a GNA affinity matrix cartridge at 1 ml/min (HP Treated). The 1918 virus used is the recombinant virus that has 2 genes (the HA and NA) of 1918 strain influenza virus along with 6 genes from Texas 91 influenza strain. The proper nomenclature or designation of the virus is 1918 HA/NA:Tx/36/91. (Tumpey T M, et al. (2005), Pathogenicity of Influenza Viruses with Genes from the 1918 Pandemic Virus: Functional Roles of Alveolar Macrophages and Neutrophils in Limiting Virus Replication and Mortality in Mice. J. Virology 79(23):14933-14944, herein incorporated by reference in its entirety). The samples were taken prior to the start and after 0, 1, 2, 4, 6 hour of recirculation. The recirculation were continued overnight (18-24 hr) for the final sample. Untreated samples to control for virus decomposition were taken at 2 and 6 hrs and overnight. The amount of viral RNA in the sample was determined before and after circulation through the cartridge by quantitative real time RT PCR using a modified version of the protocol outlined in Example 8 for use on cell culture supernatant. All PCR samples were determined in triplicate.

The collected data is presented in Table 4.

TABLE 4
1918 Flu (initial 9.4 × 10⁹ cpm)

| | HP Treated | Control |
|---|---|---|
| Time (h) | % Initial | % Initial |
| 0 | 100% | 100% |
| 1 | 37% | — |
| 2 | 24% | 75% |
| 4 | 10% | — |
| 6 | 7% | 53% |
| 20 | 0.3% | 26% |

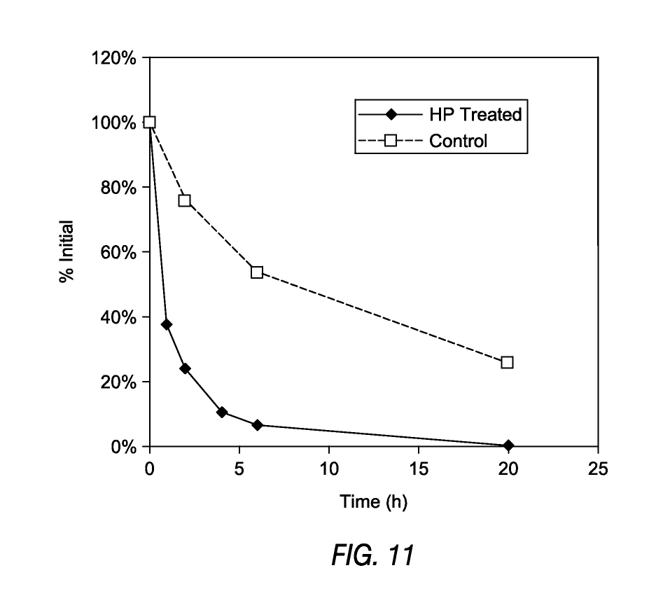

FIG. 11 is a graphical depiction of the average of the three experiments. The half-time was 55 minutes (initial 9.4×10⁹ cpm) vs ˜7 hr for the untreated benchtop control.

### Example 14

Cell culture supernatants (5 ml) from Ebola Zaire virus infected cells (10⁹ to 10¹⁰ copies/ml) were circulated through a GNA affinity matrix cartridge at 1 ml/min (HP Treated). Samples were taken prior to the start and after 0, 1, 2, 4, 6 hour of recirculation. The recirculation were continued overnight (18-24 hr) for the final sample. Untreated samples to control for virus decomposition were taken at 0, 1, 2, 4, 6 hrs and overnight. The amount of viral RNA in the sample was determined before and after circulation through the cartridge by quantitative real time RT PCR using a modified version of the protocol outlined in Example 8 for use on cell culture supernatant. All PCR samples were determined in triplicate.

The collected data is presented in Table 5.

TABLE 5
Ebola (initial 2 × 10⁹ cpm)

| | HP Treated | Control |
|---|---|---|
| Time (h) | % Initial | % Initial |
| 0 | 100% | 100% |
| 1 | 79% | 79% |
| 2 | 56% | 79% |
| 4 | 40% | 79% |
| 6 | 35% | 79% |
| 24 | 2% | 79% |

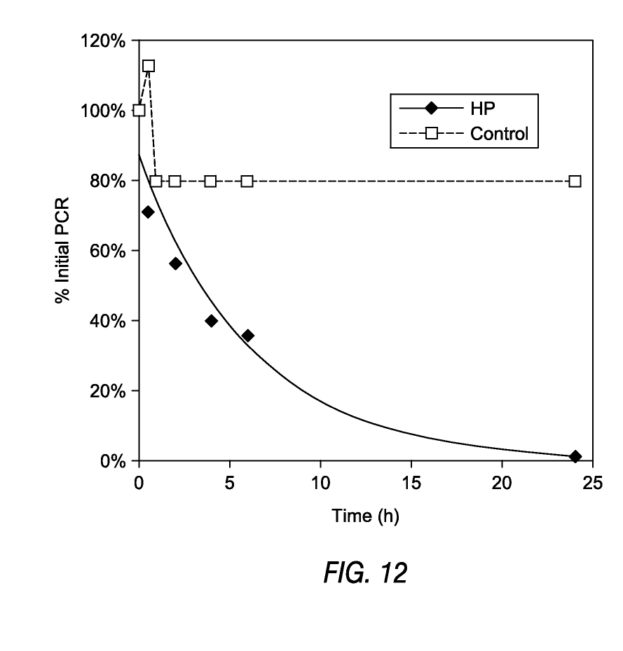

FIG. 12 is a graphical depiction of the average of the three experiments The half-time was ˜3 hr (initial 2×10⁹ cpm). The untreated benchtop control was stable after an initial 20% drop.

### Example 15

Cell culture supernatants (5 ml) from Monkeypox virus infected cells (10⁶ To 10⁷ copies/ml) were circulated through a GNA affinity matrix cartridge at 1 ml/min (HP Treated). Samples were taken prior to the start and after 0, 1, 2, 4, 6 hour of recirculation. The recirculation were continued overnight (18-24 hr) for the final sample. Untreated samples to control for virus decomposition were taken at 6 hrs and overnight. The amount of viral RNA in the sample was determined before and after circulation through the cartridge by quantitative real time RT PCR using a modified version of the protocol outlined in Example 8 for use on cell culture supernatant. All PCR samples were determined in triplicate.

The collected data is presented in Table 6.

TABLE 6
Monkeypox (initial 1.4 × 10⁶ cpm)

| | HP Treated | Control |
|---|---|---|
| Time (h) | % Initial | % Initial |
| 0 | 100% | 100% |
| 1 | 62% | nd |
| 2 | 43% | nd |
| 4 | 20% | nd |
| 6 | 10% | 73% |
| 20 | 1% | 68% |

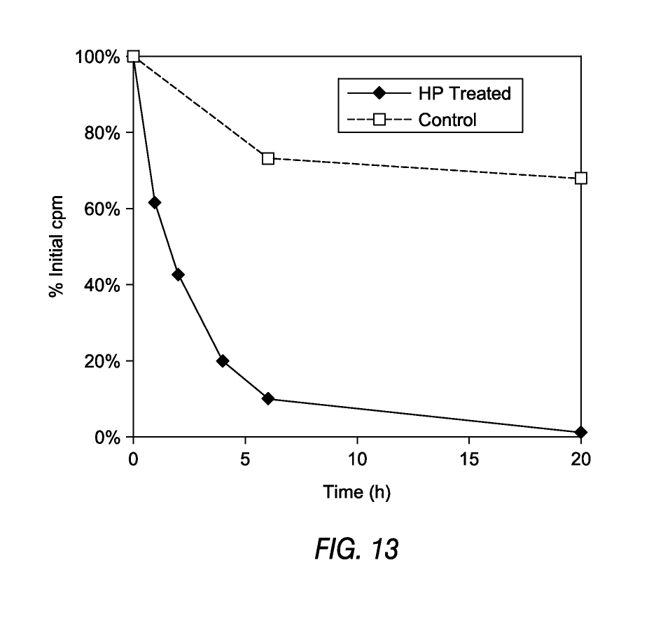

FIG. 13 is a graphical depiction of the average of the three experiments The half-time was ˜1.5 hr (initial 1.4×10⁶ cpm). The untreated benchtop control was fairly stable showing a 30% drop over 20 hours.

### Example 16

Vaccinia virus (Dryvax) in plasma was diluted into whole human blood (15 ml) and was recirculated over a Microkros miniature GNA Hemopurifier at 1 ml/min at room temperature (HP Treated). A control agarose bead filled cartridges was run as a control. Samples were analyzed for viral load by RT-PCR (in triplicate). Samples were taken prior to the start and after 0, 1, 2, 4, 6 hour of recirculation. The recirculation were continued overnight (18-24 hr) for the final sample. The amount of viral RNA in the sample was determined before and after circulation through the cartridge by quantitative real time RT PCR using the protocol outlined in Example 8. All PCR samples were determined in triplicate.

The collected data is presented in Table 7.

TABLE 7
Vaccinia (initial 40,000 cpm)

| | HP Treated | Control |
|---|---|---|
| Time | % initial | % initial |
| 0 | 100% | 100% |
| 1 | 15% | 47% |
| 2 | 10% | 70% |
| 3 | 19% | 51% |
| 6 | ND | 71% |
| 18 | 3% | 58% |

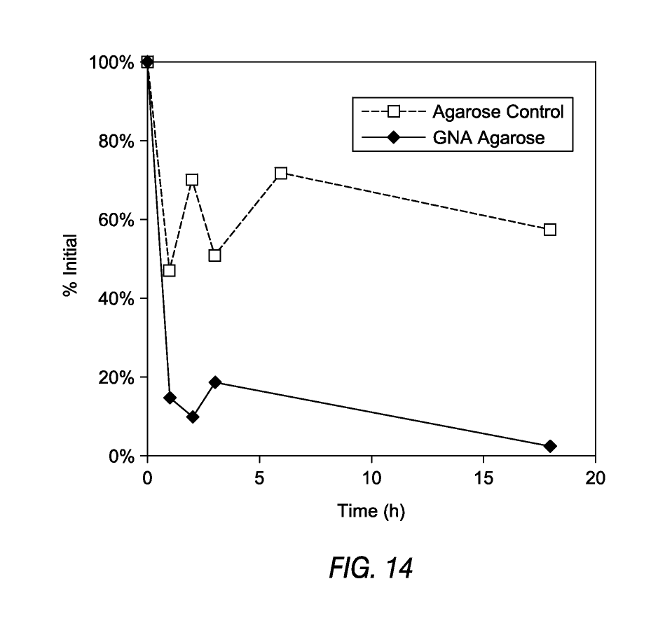

FIG. 14 is a graphical depiction of the average of the three experiments. The half-time was <1 hr (initial 4×10⁴ cpm) and >18 hr for the control.

### Example 17

Cell culture supernatants (5 ml) from West Nile Virus infected cells (10⁵ to 10⁶ copies/ml) were circulated through a GNA affinity matrix cartridge at 1 ml/min (HP Treated). Samples were taken prior to the start and after 0, 1, 2, 4, 6 hour of recirculation. The recirculation were continued overnight (18-24 hr) for the final sample. Untreated samples to control for virus decomposition were taken at 6 hrs and overnight. The amount of viral RNA in the sample was determined before and after circulation through the cartridge by quantitative real time RT PCR using a modified version of the protocol outlined in Example 8 for use on cell culture supernatant. All PCR samples were determined in triplicate.

The collected data is presented in Table 8.

TABLE 8
West Nile Virus (initial 6.7 × 10⁵)

| | HP Treated | Control |
|---|---|---|
| Time (h) | % Initial | % Initial |
| 0 | 100% | 100% |
| 1 | 89% | nd |
| 2 | 75% | nd |
| 4 | 47% | nd |
| 6 | 21% | 104% |
| 20 | 0.7% | 103% |

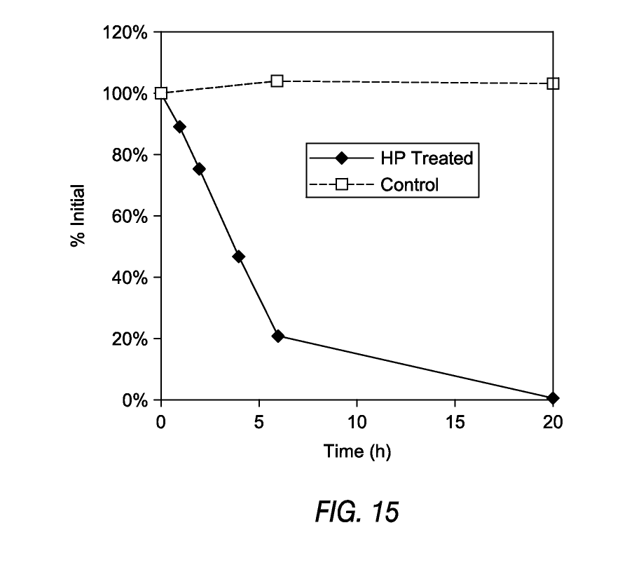

FIG. 15 is a graphical depiction of the average of the three experiments. The half-time was ˜3 hr (initial ˜6.7×10⁵ cpm). The untreated benchtop control was stable over 20 hours.

### Example 18

Blood (5 ml) from an individual infected with Ebola Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Ebola Virus is removed more efficiently than total virus RNA. The clearance of pfu of live virus from biological fluids is more pronounced than would be indicated by assays designed to measure viral load without regard to the prevalence of infectious particles.

### Example 19

Blood (5 ml) from an individual infected with Dengue Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Dengue Virus is removed more efficiently than total virus RNA. The clearance of pfu of live virus from biological fluids is more pronounced than would be indicated by assays designed to measure viral load without regard to the prevalence of infectious particles.

### Example 20

Blood (5 ml) from an individual infected with Marburg Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Marburg Virus is removed more efficiently than total virus RNA. The clearance of pfu of live virus from biological fluids is more pronounced than would be indicated by assays designed to measure viral load without regard to the prevalence of infectious particles.

### Example 21

Blood (5 ml) from an individual infected with Smallpox Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Smallpox Virus is removed efficiently from the sample.

### Example 22

Blood (5 ml) from an individual infected with Lassa Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Lassa Virus is removed more efficiently than total virus RNA. The clearance of pfu of live virus from biological fluids is more pronounced than would be indicated by assays designed to measure viral load without regard to the prevalence of infectious particles.

### Example 23

Blood (5 ml) from an individual infected with Rift Valley Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Rift Valley Virus is removed more efficiently than total virus RNA. The clearance of pfu of live virus from biological fluids is more pronounced than would be indicated by assays designed to measure viral load without regard to the prevalence of infectious particles.

### Example 24

Blood (5 ml) from an individual infected with West Nile Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious West Nile Virus is removed more efficiently than total virus RNA. The clearance of pfu of live virus from biological fluids is more pronounced than would be indicated by assays designed to measure viral load without regard to the prevalence of infectious particles.

### Example 25

Blood (5 ml) from an individual infected with H5N1 Influenza Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious H5N1 Influenza Virus is removed more efficiently than total virus RNA. The clearance of pfu of live virus from biological fluids is more pronounced than would be indicated by assays designed to measure viral load without regard to the prevalence of infectious particles.

### Example 26

Blood (5 ml) from an individual infected with Measles Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Measles Virus is removed more efficiently than total virus RNA. The clearance of pfu of live virus from biological fluids is more pronounced than would be indicated by assays designed to measure viral load without regard to the prevalence of infectious particles.

### Example 27

Blood (5 ml) from an individual infected with Mumps Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Mumps Virus is removed more efficiently than total virus RNA. The clearance of pfu of live virus from biological fluids is more pronounced than would be indicated by assays designed to measure viral load without regard to the prevalence of infectious particles.

### Example 28

Blood (5 ml) from an individual infected with an encephalitis virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious an encephalitis virus is removed more efficiently than total virus RNA. The clearance of pfu of live virus from biological fluids is more pronounced than would be indicated by assays designed to measure viral load without regard to the prevalence of infectious particles.

### Example 29

Blood (5 ml) from an individual infected with Monkeypox Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Monkeypox Virus is removed more efficiently than total virus RNA. The clearance of pfu of live virus from biological fluids is more pronounced than would be indicated by assays designed to measure viral load without regard to the prevalence of infectious particles.

### Example 30

Blood (5 ml) from an individual infected with Camelpox Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Camelpox Virus is removed more efficiently than total virus RNA. The clearance of pfu of live virus from biological fluids is more pronounced than would be indicated by assays designed to measure viral load without regard to the prevalence of infectious particles.

### Example 31

Blood (5 ml) from an individual infected with Vaccinia Virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Vaccinia Virus is removed efficiently than total virus RNA. The clearance of pfu of live virus from biological fluids is more pronounced than would be indicated by assays designed to measure viral load without regard to the prevalence of infectious particles.

### Example 32

Blood (5 ml) from an individual infected with HIV is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious HIV is removed more efficiently than total virus RNA. The clearance of pfu of live virus from biological fluids is more pronounced than would be indicated by assays designed to measure viral load without regard to the prevalence of infectious particles.

### Example 33

Blood (5 ml) from an individual infected with HCV is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious HCV is removed more efficiently than total virus RNA. The clearance of pfu of live virus from biological fluids is more pronounced than would be indicated by assays designed to measure viral load without regard to the prevalence of infectious particles.

### Example 34

Blood (5 ml) from an individual infected with a hepatitis virus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious hepatitis virus is removed more efficiently than total virus RNA. The clearance of pfu of live virus from biological fluids is more pronounced than would be indicated by assays designed to measure viral load without regard to the prevalence of infectious particles.

### Example 35

Blood (5 ml) from an individual infected with Human Cytomegalovirus is circulated through a GNA affinity matrix cartridge at 1.5 ml/min. Samples are collected prior to the start of circulation, after 30 minutes of circulation, and after 60 minutes of circulation. The amount of viral load as measured by viral RNA and pfu (infectious virus) in each collected sample is determined by real-time qRT-PCR and conventional plaque assay, respectively.

Importantly, live infectious Human Cytomegalovirus is removed more efficiently than total virus RNA. The clearance of pfu of live virus from biological fluids is more pronounced than would be indicated by assays designed to measure viral load without regard to the prevalence of infectious particles.

### Example 36

Preparation of the Device:

The lectin affinity viral hemodialysis device is made by pouring a dry powder consisting of GNA immobilized on diatomaceous earth (CHROMOSORB GAW 60/80; Celite Corp, Lompoc, Calif.) into the outside compartment of a hollow-fiber plasmapheresis column (PLASMART 60; Medica, srl, Medollo Italy) using a funnel attached to the outlet ports of the column. The powder (40 grams) is introduced under gravity flow with shaking to fill the available extrafiber space. For therapeutic use, the cartridges containing the affinity resin is heat sealed in TYVEK shipping pouches and sterilized with 25-40 kGy gamma irradiation. Samples of the product are then tested for sterility and endotoxin and found to meet FDA standards. The finished product can be stored for at least 6 months at room temperature in a cool dry place until ready for use.

Preparation for Treatment:

The hemodialysis cartridge is opened under aseptic conditions and placed in line on an appropriate blood pumping system (e.g. COBE C3 plus hemodialysis machine). The cartridge is then flushed with at least 1 liter of sterile saline. During this procedure, all bubbles are removed from the tubing and the cartridge by gentle tapping.

Treatment:

For use on a patient with established vascular access, the patient is connected to the dialysis machine, which pumps blood from the patient through the cartridge and returns the purified blood to the patient. Blood flow rates are typically maintained at 200 to 400 ml/min at the discretion of the attending physician. Heparin injections are most often used to prevent blood clotting. Typical treatment times are up to 4 hours for dialysis patients. Longer times may be used to increase the effectiveness of the treatment. At the end of the treatment, the blood in the tubing and cartridge is washed back into the patient using sterile saline. The machine is then disconnected from the patient and the contaminated cartridge and blood tubing properly disposed.

Results:

The blood of a patient infected with a virus who is treated in the above manner has a significantly reduced viral load and/or pfu/ml compared with levels before treatment. Preferably, the viral load and/or pfu/ml is reduced a therapeutically effective amount.

### Example 37

The ability of lectins to remove vaccinia virus was tested. GNA was covalently coupled to aminosilane derivatized diatomaceous earth (CHROMOSORB; Celite Corp, Lompoc, Calif.) using glutaraldehyde to form the Schiff's base and cyanoborohydride to reduce the Schiffs base to a stable imine. This affinity resin was packed into single use hollow-fiber plasmapheresis cartridges (MICROKROS, Spectrum Labs, Rancho Dominguez, Calif.) for testing. Samples containing the appropriate virus were recirculated over the GNA hemodialysis device column at room temperature and test samples removed at intervals for virus determination. The GNA hemodialysis cartridge efficiently removed Vaccinia virus from aqueous buffer (>99% in 1 hour). The GNA hemodialysis device was also effective in removing vaccinia from blood as measured by real time PCR.

From the foregoing, it will be obvious to those skilled in the art the various modifications in the above-described methods, devices and compositions can be made without departing from the spirit and scope of the invention. Accordingly, the invention can be embodied in other specific forms without departing from the spirit or essential characteristics thereof. Present examples and embodiments, therefore, are to be considered in all respects as illustrative and not restrictive, and all changes which come within the meaning and range of equivalency of the claims are therefore intended to be embraced therein.

1. (canceled)

2. A method for reducing the amount of hemorrhagic virus, hemorrhagic viral particles, or hemorrhagic viral glycoproteins in blood or plasma from an individual infected with a hemorrhagic virus, said method comprising:
selecting an individual infected with a hemorrhagic virus;
providing a lectin affinity device comprising:
a processing chamber configured to receive blood or plasma comprising a hemorrhagic virus;
a lectin attached to a substrate disposed within said processing chamber; and
a porous membrane, wherein said porous membrane has a pore size that allows the passage of intact hemorrhagic virus, hemorrhagic viral particles, or hemorrhagic viral glycoproteins through said pores, wherein said pore size excludes blood cells from passing through said pores, and wherein said membrane is configured in said processing chamber such that when blood or plasma comprising hemorrhagic virus, hemorrhagic viral particles, or hemorrhagic viral glycoproteins is disposed in said processing chamber, the hemorrhagic virus, hemorrhagic viral particles, or hemorrhagic viral glycoproteins pass through said porous membrane and contact said lectin and are bound thereto, and wherein blood cells are prevented from passing through said porous membrane and are prevented from contacting said lectin; and
contacting said blood or plasma from said individual infected with said hemorrhagic virus, hemorrhagic viral particles, or hemorrhagic viral glycoproteins with said affinity device.

3. The method of claim 2, wherein said processing chamber further comprises an inlet port and an outlet port;
wherein said porous membrane comprises one or more porous hollow fiber membranes and wherein a channel of said hollow fiber membranes is in fluidic communication with said inlet and said outlet ports;
said device having an extrachannel space within said chamber which surrounds said hollow fiber membranes; and
wherein said lectin is attached to a substrate that is disposed within said extrachannel space proximate to an exterior surface of said membranes, wherein said lectin binds said hemorrhagic virus, hemorrhagic viral particles, or hemorrhagic viral glycoproteins and traps said hemorrhagic virus, hemorrhagic viral particles, or hemorrhagic viral glycoproteins in the extrachannel space.

4. The method of claim 3, wherein at least 50% of said hemorrhagic virus, hemorrhagic viral particles, or hemorrhagic viral glycoproteins in blood or plasma are removed from said blood or plasma.

5. The method of claim 3, wherein following said contacting said blood or plasma with said device, no greater than 1×10⁴ pfu/ml hemorrhagic virus, hemorrhagic viral particles, or hemorrhagic viral glycoproteins remain in said blood or plasma.

6. The method of claim 3, wherein the amount of hemorrhagic virus, hemorrhagic viral particles, or hemorrhagic viral glycoproteins in said blood or plasma is reduced to a clinically relevant amount.

7. The method of claim 3, wherein said blood or plasma is exposed to said lectin for no longer than 360 minutes.

8. The method of claim 3, wherein said blood or plasma is exposed to said lectin for no longer than 90 minutes.

9. The method of claim 3, wherein said membrane comprises pores about 200-500 nm in diameter.

10. The method of claim 3, wherein said substrate is selected from the group consisting of agarose, aminocelite, resin, silica, polysaccharide, plastic, and protein.

11. The method of claim 3, wherein said lectin is linked to said substrate by a linker.

12. The method of claim 11, wherein said linker is selected from the group consisting of avidin, streptavidin, biotin, protein A, protein G, gluteraldehyde, C₂ to C₁₈ dicarboxylates, diamines, dialdehydes, and dihalides or mixtures thereof.

13. The method of claim 3, wherein said lectin is selected from the group consisting of Galanthus nivalis agglutinin (GNA), Narcissus pseudonarcissus agglutinin (NPA), cyanovirin, and Concanavalin A or mixtures thereof.

14. The method of claim 3, wherein said lectin is GNA.

15. The method of claim 2, wherein the hemorrhagic virus, hemorrhagic viral particles, or hemorrhagic viral glycoproteins is an Ebola virus.

16. The method of claim 2, wherein the hemorrhagic virus, hemorrhagic viral particles, or hemorrhagic viral glycoproteins is a Dengue virus.

17. The method of claim 3, wherein the hemorrhagic virus, hemorrhagic viral particles, or hemorrhagic viral glycoproteins is an Ebola virus.

18. The method of claim 3, wherein the hemorrhagic virus, hemorrhagic viral particles, or hemorrhagic viral glycoproteins is a Dengue virus.

19. A method of treating an individual infected with a lectin-binding virus by rapidly reducing the amount of hemorrhagic virus, hemorrhagic viral particles, or hemorrhagic viral glycoproteins in the blood of said individual, said method comprising:
identifying an individual infected with a lectin-binding virus;
removing blood from said individual;
providing a lectin affinity device comprising:
a processing chamber configured to receive blood or plasma contaminated with viral plaque forming units;
lectin attached to a substrate disposed within said processing chamber; and
a porous membrane wherein said membrane has a pore size to allow passage of intact hemorrhagic virus through said pores and wherein said pore size excludes blood cells from passing through said pores, said porous membrane configured in said processing chamber such that when blood or plasma contaminated with hemorrhagic virus, hemorrhagic viral particles, or hemorrhagic viral glycoproteins is disposed in said processing chamber, said hemorrhagic virus pass through said membrane and contact said lectin and are bound thereto, and wherein blood cells are prevented from passing through said membrane and are prevented from contacting said lectin;
transferring said blood into said chamber such that said hemorrhagic virus, hemorrhagic viral particles, or hemorrhagic viral glycoproteins contact said lectin and are bound thereto;
removing said blood from said chamber; and
returning said removed blood into said individual, wherein said blood is exposed to said lectin for no longer than 360 minutes.

20. The method of claim 19, further comprising repeating said removing, transferring, and returning steps until a volume of blood equivalent to at least about the total blood volume of said individual has been exposed to said lectin.

21. The method of claim 18, further comprising repeating said removing, transferring, and returning steps until at least 50% of said hemorrhagic virus, hemorrhagic viral particles, or hemorrhagic viral glycoproteins are removed from said individual's blood.

22. The method of claim 18, further comprising repeating said removing, transferring, and returning steps until the concentration of hemorrhagic virus, hemorrhagic viral particles, or hemorrhagic viral glycoproteins in said individual's blood is no greater than 1×10⁴ pfu/ml.
